(19) United States
(12) Patent Application Publication
(10) Pub. No.: US 2022/0120768 A1
(43) Pub. Date: Apr. 21, 2022

(54) REAGENT FOR MEASURING FIBRINOGEN

(71) Applicant: A&T CORPORATION, Fujisawa-shi, Kanagawa (JP)

(72) Inventors: Takashi NISHIYAMA, Fujisawa-shi, Kanagawa (JP); Kazuya NAKAMOTO, Fujisawa-shi, Kanagawa (JP); Masayoshi KIKUCHI, Fujisawa-shi, Kanagawa (JP); Takayoshi TORAMI, Fujisawa-shi, Kanagawa (JP); Shuichi OKA, Fujisawa-shi, Kanagawa (JP)

(21) Appl. No.: 17/311,131

(22) PCT Filed: Dec. 5, 2019
(86) PCT No.: PCT/JP2019/047592

§ 371 Date: Oct. 21, 2021

(30) Foreign Application Priority Data
Dec. 7, 2018 (JP) 2018-229919
Apr. 12, 2019 (JP) 2019-076180

(51) Int. Cl. G01N 33/86

(52) U.S. Cl. CPC G01N 33/86; G01N 2800/224

(57) ABSTRACT

The present disclosure provides a dry reagent and a method that enable fibrinogen determination without dilution of the sample. More specifically, the present disclosure provides a fibrinogen measurement dry reagent of an undiluted sample comprising: (i) thrombin or a protein having thrombin activity; (ii) magnetic particles; (iii) a fibrin monomer polymerization inhibitor; (iv) a calcium salt; (v) a dry reagent layer solubility improving agent; (vi) a dry reagent layer reinforcing material; and (vii) a buffer and a method for fibrinogen determination.

### TECHNICAL FIELD

The present invention relates to a fibrinogen measurement reagent and a fibrinogen determination method using the same.

### BACKGROUND ART

Fibrinogen plays a key role in the blood coagulation cascade and hemostasis.

Determination of fibrinogen concentration is a test intended to examine the normality/abnormality in blood clotting ability along with prothrombin time (also referred to as “PT”) and activated partial thromboplastin time (also referred to as “APTT”), and is extensively performed in clinical practice and, in particular, in clinical laboratories.

Examples of techniques that can readily determine fibrinogen concentration by adding a sample to a dry reagent card dropwise include the fibrinogen measurement dry reagent disclosed in Patent Literature 1 and the fibrinogen determination method disclosed in Patent Literature 2. The fibrinogen measurement dry reagent disclosed in Patent Literature 1 involves the use of diluted blood plasma. The method disclosed in Patent Literature 2 requires sample preparation and a whole blood sample is diluted to 7.5- to 10-fold, a plasma sample is diluted to 15-fold, and then the sample is added to a reagent card dropwise. However, when fibrinogen is to be analyzed urgently in, for example, a delivery room or operating room or at bed side, it is difficult to use a system that requires a dilution procedure as an essential procedure.

On the other hand, examples of techniques that enable fibrinogen determination with an undiluted sample include the method disclosed in Patent Literature 3. According to the method disclosed in Patent Literature 3, use of an undiluted sample involves the use of an excessive amount of thrombin, so as to convert all fibrinogens into fibrin monomers. Further, in order to suppress the polymerization reaction of the resulting fibrin monomers and prolong the clotting time, a fibrin monomer polymerization inhibitor (G-P-R-P-A-amide) is used. According to the method disclosed in Patent Literature 3, it is necessary to dissolve reagents in purified water in advance to prepare a liquid reagent and incubate the reagent until immediately before the measurement. In addition, calibration is necessary before the measurement. That is, it is difficult to apply the method disclosed in Patent Literature 3 to urgent fibrinogen determination because of the need of incubation of the dissolved reagent or calibration. The technique disclosed in Patent Literature 3 does not adopt a dry reagent card system. Further, and in general, a composition suitable for a reagent to be reacted in a liquid state is different from a composition suitable for a dry reagent card.

In recent years, importance of fibrinogen determination has been pointed out in the perioperative period and perinatal period. In the case of critical bleeding, fibrinogen concentration in the blood becomes significantly low. If fibrinogen concentration in a patient's blood is measured to be less than 150 mg/dl, then, fresh frozen plasma or a concentrated fibrinogen preparation is administered to the patient for life support. Further, after fresh frozen plasma or a concentrated fibrinogen preparation is administered, it is necessary to examine as to whether or not fibrinogen concentration in the blood has returned to the normal range. When fibrinogen concentration in the blood has not reached the normal range after the treatment, further treatment becomes necessary for patient's life support and, therefore, this measurement needs to be performed promptly.

In the perioperative period and perinatal period, fibrinogen determination is used for the purposes as described above and, therefore, a system that can measure fibrinogen concentration in the blood with higher promptness and accuracy has been desired.

In the fibrinogen determination method involving the use of a thrombin reagent solution, the thrombin time method developed by Clauss VA is generally employed (Clauss VA, Gerinnungsphysiologische schnellmethode zur bestimmung des fibrinogens, ActaHaematologica, 17, 237-246, 1957). The thrombin time method utilizes the characteristic that the rate for fibrinogen conversion into fibrin under excessive amount of thrombin predominantly depends on the fibrinogen concentration.

This determination method comprises diluting blood plasma in a buffer, preheating the diluted solution, adding a thrombin-containing reagent solution, measuring the clotting time, and converting the obtained clotting time into fibrinogen concentration using a calibration curve prepared in advance. The clotting time according to this determination method is the period from the addition of the thrombin reagent solution to the end point. The end point is detected via an optical measurement that detects an increase in turbidity or via a physical measurement that detects an increase in viscosity.

The determination method described above and thrombin reagents used for the determination method are extensively accepted in the world and employed in clinical laboratories. However, such determination method was not necessarily suitable for use in the perioperative period and perinatal period in the following respect. For example, it is necessary to reconstitute a lyophilized thrombin reagent with purified water or the like every time for each measurement (a reconstituted solution cannot be stored over a long period of time), whole blood must be centrifuged to separate plasma therefrom, plasma must be diluted with a diluent, and the diluted plasma solution must be preheated. That is, such determination method may not necessarily have been ideal because it takes a long time before the measurement and involves a large number of steps.

An example of an improved version of the fibrinogen determination method described above is a fibrinogen determination method using a thrombin-containing dry reagent. Such method is disclosed in JP H06-094725 A (JP Patent No. 2776488) and JP H06-141895 A (JP Patent No. 2980468). A thrombin-containing dry reagent used for such determination method is prepared by adding magnetic particles to a thrombin reagent solution, dispensing a given amount of the mixture on a reaction slide, and then lyophilizing the mixture.

The determination method involving the use of such dry reagent is characterized in that, after the addition of the sample to the reagent, a combination of an oscillating magnetic field and a static permanent magnetic field is applied at a given interval, magnetic particles contained in the dry reagent are allowed to move (physically), the movement signal of the magnetic particles are detected as the amount of change in the scattered light, and the end point is detected based on the amount of change with the elapse of time (i.e., as time elapses). The period from the addition of the reagent to the end point is designated as the clotting time, and the obtained clotting time is converted into the fibrinogen concentration with a calibration curved prepared in advance.

An example of an analyzer that can implement such method is CG02N (product name; commercialized by A&T Corporation). With the use of such analyzer, a combination of an oscillating magnetic field and a static permanent magnetic field is applied at intervals of 0.5 seconds with the elapse of time, and the movement signal of the magnetic particles are monitored at the same interval.

When the analyzer described above is used, the change in the movement signal of the magnetic particles as time elapses inversely corresponds (is inversely correlated) to the change in the viscosity of the dry reagent. The end point is detected as the point at which the movement signal of the magnetic particles is attenuated by 30% from the peak value of the movement signal of the magnetic particles. The peak value of the movement signal of the magnetic particles obtained immediately after addition of the sample is the point at which the constituents of the dry reagent are completely dissolved. i.e., the point at which the viscosity in the dry reagent becomes the lowest. Let the peak value of the movement signal be designated as X and the signal intensity after a given period of time thereafter be designated as Y. Then, the increase in the viscosity at the time point when the attenuation in signal intensity is (X−Y)×100/X (%), is equivalent to the point at which the viscosity is X/Y times the minimal viscosity. That is, the point at which the movement signal of the magnetic particles is attenuated by 30% from the peak value of the movement signal is equivalent to the point at which the viscosity is increased to 1.43 times the minimal viscosity after the addition of the sample.

In JP H06-141895 A (JP Patent No. 2980468), the technique described above is described as a fibrinogen determination method comprising mixing a fibrinogen measurement dry reagent comprising a protein having thrombin activity and magnetic particles with a sample, and measuring the clotting time to determine fibrinogen concentration in the sample. In such determination method, the point at which the viscosity in the dry reagent is increased to from 1.05 times to 2.00 times the minimal viscosity is designated as the end point, and the period from the addition of the sample to the end point is designated as the clotting time.

This method is advantageous in that it is not necessary to reconstitute a lyophilized thrombin reagent with purified water or the like every time for each measurement, nor is it necessary to preheat the diluted sample. However, according to this determination method, it was necessary to dilute plasma and whole blood samples with a dedicated-purpose diluent. Thus, such determination method may not necessarily have been optimal in some aspects as a method to be employed in the perioperative period and perinatal period.

When fibrinogen concentration is determined with the thrombin-containing dry reagent disclosed in JP H06-094725 A (JP Patent No. 2776488), the clotting time obtained upon measuring undiluted plasma or undiluted whole blood is shortened to an extreme extent, and it is not possible to detect the clotting time corresponding to the fibrinogen concentration in the blood. In order to detect the clotting time corresponding to the fibrinogen concentration in the blood, accordingly, it was necessary to prolong (extend) the clotting time.

While a large number of documents including academic articles, patent applications, and manufacturers' instructions are cited herein, such disclosures are not to be deemed as being related to the patentability of the present invention.

### PRIOR ART LITERATURES

### Patent Literatures

Patent Literature 1: JP H06-094725 A (JP Patent No. 2776488)
Patent Literature 2: JP H06-141895 A (JP Patent No. 2980468)
Patent Literature 3: JP H05-219993 A (JP Patent No. 3469909) Non-Patent Literatures
Non-Patent Literature 1: Clauss VA: Gerinnungsphysiologische schnellmethode zur bestimmung des fibrinogens, Acta Haematologica, 17, 237-246, 1957

### SUMMARY OF THE INVENTION

An object of the present disclosure is to provide a fibrinogen measurement reagent that can readily determine the fibrinogen concentration in an undiluted sample with high reproducibility and accuracy, so as to solve at least part of the problems described above.

The present inventors have conducted concentrated studies in order to solve the problems described above. As a result, the present inventors found that the problems described above can be solved with the fibrinogen measurement dry reagent according to the present disclosure, thereby completing the present invention encompassing such findings as an embodiment.

Further, in order to solve at least part of the problems described above, an object of the present disclosure is to provide a novel technique that can be implemented in the perioperative period and perinatal period. More specifically, an object of the present disclosure is to provide a fibrinogen determination method that can be implemented with high accuracy using of a fibrinogen measurement dry reagent which does not require a dilution procedure of the plasma sample or whole blood sample.

More specifically, the present inventors have completed the fibrinogen measurement dry reagent described above by including an amino acid or salt thereof or a saccharide to improve the solubility of the dry reagent, including highly active thrombin or a highly active thrombin-like protein to strongly accelerate the thrombin reaction, and including a fibrin monomer polymerization inhibitor to inhibit self-assembly of fibrin monomers. With such fibrinogen measurement dry reagent, the clotting time can be prolonged (extended) without adversely affecting the reproducibility of the clotting time.

However, when the present inventors applied the dry reagent mentioned above to the determination method described in JP H6-141895 A (JP Patent No. 2980468), while undiluted plasma was determined accurately, undiluted whole blood could not always be accurately determined even when hematocrit correction was carried out. Without wishing to be bound to any particular theory, it is believed that in the case of undiluted whole blood measurements, such inaccuracy may be caused by variability of the solubility of the fibrinogen measurement dry reagent depending on viscosity differences among samples, in addition to the variability of the amount of plasma components depending on hematocrit value differences of the samples. In the case of undiluted whole blood measurements, it was found that fibrinogen determination may not be performed correctly without defining the starting point (i.e., the starting point of the coagulation reaction: the measurement time of 0 (sec)). Such problem was not recognized in the prior art. In order to solve at least part of these problems, a further object of the present disclosure is to provide a novel technique that can be implemented in the perioperative period and perinatal period; i.e., a fibrinogen determination method that can be implemented with high accuracy with a fibrinogen measurement dry reagent which does not require a dilution procedure of the plasma sample or whole blood sample.

The present inventors have conducted concentrated studies in order to solve the problems described above. As a result, the present inventors found that the starting point (i.e., the starting point of the coagulation reaction: the measurement time 0 (sec)) can be determined by analyzing the movement signal of the magnetic particles detected after the addition of the samples to the fibrinogen measurement dry reagent. More specifically, the present inventors found that the starting point (start point) can be detected by computing a plurality of ratios of the movement signals of the magnetic particles at a given time interval, and detecting a point within an interval during which such ratio is maintained within a given range for a given period of time, thereby completing the present invention encompassing such finding as an embodiment.

The present disclosure encompasses the following embodiments.

### Embodiment 1

A fibrinogen measurement dry reagent for use in measuring an undiluted whole blood or plasma sample comprising:

(i) thrombin or a protein having thrombin activity;

(ii) magnetic particles:

(iii) a fibrin monomer polymerization inhibitor:

(iv) a calcium salt;

(v) a dry reagent layer solubility improving agent;

(vi) a dry reagent layer reinforcing material: and

(vii) a buffer.

### Embodiment 2

The fibrinogen measurement dry reagent of Embodiment 1, wherein the thrombin or the protein having thrombin activity is bovine thrombin.

### Embodiment 3

The fibrinogen measurement dry reagent of Embodiment 1 or 2, wherein the magnetic particles are triiron tetraoxide particles.

### Embodiment 4

The fibrinogen measurement dry reagent of any of Embodiments 1 to 3, wherein the inhibitor of fibrin monomer polymerization is GPRP-amide or GHRP-amide.

### Embodiment 5

The fibrinogen measurement dry reagent of any of Embodiments 1 to 4, wherein the calcium salt is calcium chloride dihydrate.

### Embodiment 6

The fibrinogen measurement dry reagent of any of Embodiments 1 to 5, wherein the agent for improving solubility of the dry reagent layer is glycine.

### Embodiment 7

The fibrinogen measurement dry reagent of Embodiment 6, which comprises 1.5% to 4.0% by weight of glycine in the final solution.

### Embodiment 8

The fibrinogen measurement dry reagent of any of Embodiments 1 to 7, wherein the material for reinforcing the dry reagent layer is bovine serum albumin.

### Embodiment 9

The fibrinogen measurement dry reagent of any of Embodiments 1 to 8, wherein the buffer is HEPES-NaOH buffer.

### Embodiment 10

The fibrinogen measurement dry reagent of any of Embodiments 1 to 9, which further comprises a heparin neutralizer and/or a defoaming agent.

### Embodiment 11

The fibrinogen measurement dry reagent of Embodiment 10, wherein the heparin neutralizer is polybrene and/or the defoaming agent is sorbitan monolaurate.

### Embodiment 12

A fibrinogen determination method comprising:

(i) a step of adding a sample to a fibrinogen measurement dry reagent containing magnetic particles;

(ii) a step of allowing the magnetic particles in the reagent to move after the addition of the sample and monitoring the movement signal of the magnetic particles, and

(iii) a step of computing a plurality of ratios of the movement signals of the magnetic particles monitored in step (ii) at a given time interval,

wherein a point within an interval during which the ratio of the movement signals of the magnetic particles monitored at the given time interval is maintained within a given range for a given period of time is designated as the starting point, a point at or after the starting point at which the movement signal of the magnetic particles is attenuated by 5% to 50% from the peak value of the movement signal of the magnetic particles is designated as the end point, and the time from the starting point to the end point is designated as the clotting time.

### Embodiment 13

The fibrinogen determination method of Embodiment 12, wherein the time interval used to compute the ratio of the movement signals of the magnetic particles is a given time interval selected from between 0.1 seconds and 2 seconds.

### Embodiment 14

The fibrinogen determination method of Embodiment 12 or 13, wherein the time interval used to compute the ratio of the movement signals of the magnetic particles is a time interval of 0.5 seconds, 1 second, 1.5 seconds, or 2 seconds.

### Embodiment 15

The fibrinogen determination method of Embodiment 12 or 13, wherein the time interval used to compute the ratio of the movement signals of the magnetic particles is a time interval of 1 second.

### Embodiment 16

The fibrinogen determination method of Embodiment 12, wherein the given range of the ratio of the movement signals of the magnetic particles is 1.0±0.2.

### Embodiment 17

The fibrinogen determination method of Embodiment 13, wherein the given range of the ratio of the movement signals of the magnetic particles is 1.0±0.1.

### Embodiment 18

The fibrinogen determination method of any of Embodiments 12 to 17, wherein the time period during which the ratio of the movement signals of the magnetic particles is maintained within a given range is 1.5 seconds.

### Embodiment 19

The fibrinogen determination method of any of Embodiments 12 to 18, wherein the first point of the time period during which the ratio of the movement signals of the magnetic particles is maintained within a given range is designated as the starting point.

### Embodiment 20

The fibrinogen determination method of any of Embodiments 12 to 19, wherein a point at or after the starting point at which the movement signal of the magnetic particles is attenuated by 20% to 30% from the peak value of the movement signal of the magnetic particles is designated as the end point.

### Embodiment 21

The fibrinogen determination method of Embodiment 20, wherein a point at or after the starting point at which the movement signal of the magnetic particles is attenuated by 30% from the peak value of the movement signal of the magnetic particles is designated as the end point.

### Embodiment 22

The fibrinogen determination method of Embodiment 20, wherein a point at or after the starting point at which the movement signal of the magnetic particles is attenuated by 20% from the peak value of the movement signal of the magnetic particles is designated as the end point.

### Embodiment 23

A program for executing the fibrinogen determination method of any of Embodiments 12 to 22.

### Embodiment 24

An information recording medium comprising the program of Embodiment 23 recorded thereon.

### Embodiment 25

An apparatus for fibrinogen determination comprising the program of Embodiment 23 integrated therein or the information recording medium of Embodiment 24 stored therein. This description includes the content as disclosed in the descriptions and/or drawings of Japanese Patent Application Nos. 2018-229919 and 2019-076180, which are priority documents of the present application.

### Advantageous Effects of the Invention

According to the present disclosure, fibrinogen can be determined with high accuracy without the need for preparation of a reagent or dilution of a sample.

### BRIEF DESCRIPTION OF THE DRAWINGS

FIG. 1 shows an example of a representative reaction slide used for a fibrinogen measurement dry reagent.

FIG. 2 is a partially exploded diagram of the reaction slide shown in FIG. 1.

FIG. 3 shows the results of the correlation test between the fibrinogen concentration in plasma and the clotting time performed in Example 1. The linear relationship between the fibrinogen concentration in plasma and the clotting time is shown.

FIG. 4 shows the results of the correlation test between the results obtained by Clauss's method (the thrombin time method developed by Clauss VA; reference: Gerinnungsphysiologische schnellmethode zur bestimmung des fibrinogens, Acta Haematologica, 17, 237-246, 1957) and the results obtained with the reagent of the present disclosure performed in Example 3 (the correlation with the conventional method).

FIG. 5 shows the results of the correlation test between the results of plasma measurements and the results of whole blood measurements using the reagent of the present disclosure performed in Example 4 (the correlation between types of samples).

FIG. 6 shows changes in the movement signal of the magnetic particles with the elapse of time when measured using the reagent of the present disclosure (the fibrinogen measurement dry reagent of the present disclosure).

FIG. 7 shows changes in the movement signal of the magnetic particles with the elapse of time when measured using a lyophilized reagent prepared in accordance with the reagent composition according to a conventional technique.

FIG. 8 shows photographs of the appearance of dry reagent cards before and after plasma measurements.

FIG. 9 shows a calibration curve obtained by a conventional determination method performed in Example 7 (Comparative Example 2).

FIG. 10 shows a calibration curve obtained by the determination method of the present disclosure performed in Example 7 (the present disclosure).

FIG. 11 shows the results of the correlation test between the quantitative value of fibrinogen determined by Clauss's method and the quantitative value of fibrinogen determined by the determination method of the present disclosure performed in Example 8. That is, FIG. 11 shows the correlation with Clauss's method (plasma measurements).

FIG. 12 shows the results of the correlation test between the quantitative value of fibrinogen determined using a citrated plasma sample and the quantitative value of fibrinogen determined using a citrated whole blood sample performed in Example 9 (the correlation between types of samples).

FIG. 13 shows an example concerning the determination method of the present disclosure in which the period for monitoring the movement signal of the magnetic particles, the period for computing the ratio of the movement signals of the magnetic particles, and the time interval used to compute the ratio of the movement signals of the magnetic particles are the same.

FIG. 14 shows an example concerning the determination method of the present disclosure in which the period for monitoring the movement signal of the magnetic particles and the period for computing the ratio of the movement signals of the magnetic particles are the same while the time interval used to compute the ratio of the movement signals of the magnetic particles is different.

FIG. 15 shows an example concerning the determination method of the present disclosure in which the period for monitoring the movement signal of the magnetic particles, the period for computing the ratio of the movement signals of the magnetic particles, and the time interval used to compute the ratio of the movement signals of the magnetic particles are all different from one another.

FIG. 16 shows an example concerning the determination method of the present disclosure in which the periods for monitoring the movement signal of the magnetic particles vary.

FIG. 17 shows an example concerning the determination method of the present disclosure in which the period for monitoring the movement signal of the magnetic particles and the period for computing the ratio of the movement signals of the magnetic particles vary.

FIG. 18 shows an example concerning the determination method of the present disclosure in which the ratio of the movement signals of the magnetic particles is continuously computed and then intermittently computed.

FIG. 19 shows an example concerning the determination method of the present disclosure in which the ratio of the movement signals of the magnetic particles is intermittently computed and then continuously computed.

### EMBODIMENTS OF THE INVENTION

Hereafter, the present disclosure is described with reference to the drawings.

In one embodiment, the present disclosure provides a fibrinogen determination method that can be performed in the perinatal period and perioperative period. In another embodiment, the present disclosure provides a fibrinogen determination method comprising: (i) a step of adding a sample to a fibrinogen measurement dry reagent containing magnetic particles: (ii) a step of allowing the magnetic particles in the reagent to move (physically) after the addition of the sample and monitoring the movement signal of the magnetic particles: and (iii) a step of computing the ratio of the movement signals of the magnetic particles monitored in step (ii) at a given time interval. A plurality of ratios of the movement signals of the magnetic particles at the given time interval can be computed. A point within an interval during which the ratio of the movement signals of the magnetic particles monitored at a given time interval is maintained within a given range for a given period of time can be designated as the starting point; a point at or after the starting point at which the movement signal of the magnetic particles is attenuated by 5% to 50% from the peak value of the movement signal of the magnetic particles can be designated as the end point; and the time (period) from the starting point to the end point can be designated as the clotting time. Step (ii) may be performed simultaneously with step (iii).

The phrase “the movement signal of the magnetic particles” used herein refers to the amount of change in the intensity of scattered light determined by, after the addition of the sample, applying a combination of an oscillating magnetic field and a static permanent magnetic field at a given interval, allowing the magnetic particles contained in the reagent to move, and applying light thereto in step (ii)(the same may be referred to as “Sₙ” herein). For convenience of description, the movement signal of the magnetic particles detected at the time point when the sample is added is designated as “S₀” herein.

The phrase “the time point for monitoring the movement signal of the magnetic particles” used herein refers to the time point at which the movement signal of the magnetic particles is measured (the same may be referred to as “mmₙ” herein). In the figures, the time point for monitoring the movement signal of the magnetic particles may be indicated by a solid circle. For the convenience of description, the time point for sample addition is designated as 0 second (mm₀) to define the time point for monitoring the movement signal of the magnetic particles herein. It is merely designated to define the time point, and the time point for sample addition may appropriately be set up, for example, −5 seconds, provided that the clotting time is computed by the method of the present disclosure. In some embodiments, the movement signal of the magnetic particles may be monitored continuously or intermittently.

The phrase “period for monitoring the movement signal of the magnetic particles” used herein refers to the time interval of the monitoring of the movement signal of the magnetic particles. When the time points for monitoring the movement signal of the magnetic particles S₀, S₁, S₃, S₄ . . . are designated mm₀, mm₁, mm₂, mm₃, mm₄ . . . , for example, then, the period for monitoring the movement signal of the magnetic particles can be indicated as (mm₁−mm₀), (mm₂−mm₁), (mm₃−mm₂), (mm₄−mm₃) . . . . In some embodiments, the period for monitoring the movement signal of the magnetic particles may be constant. In other embodiments, the period for monitoring the movement signal of the magnetic particles may be altered. In the figures, the period for monitoring the movement signal of the magnetic particles may be indicated by arrows (←→). In some embodiments, the period for monitoring the movement signal of the magnetic particles can be selected from between 0.1 seconds and 2 seconds.

In step (iii) of the method of the present disclosure, with regard to the movement signal of the magnetic particles monitored in step (ii), the ratio of the movement signals of the magnetic particles of a given time interval is computed. The phrase “time point for computing the ratio of the movement signals of the magnetic particles” used herein refers to the time point at which the ratio of the movement signals of the magnetic particles is computed (the same may be referred to as “mrₙ” herein). In the figures, the time point for computing the ratio of the movement signals of the magnetic particles may be indicated by an outlined circle. With regard to the apparatus of the present disclosure, for example, the movement signal of the magnetic particles is measured at the time when the sample is added (S₀), the second movement signal of the magnetic particles is measured (S₁), and from this time point and on the ratio of the movement signals of the magnetic particles (S₁/S₀) can then be computed. Such time point at which the ratio of the movement signals of the magnetic particles becomes computable is referred to herein as “the time point for computing the ratio of the movement signals of the magnetic particles”. In practice, because calculation is performed by an apparatus, there is a slight time lag and the time point at which S₁ was measured is different from (not strictly identical to) the time point for computing the ratio of the movement signals of the magnetic particles mr₁. However, for the convenience of description, the time point at which the ratio of the movement signals of the magnetic particles becomes computable is designated herein as the time point for computing the ratio of the movement signals of the magnetic particles. Incidentally, this does not mean the apparatus must immediately compute the ratio of the movement signals of the magnetic particles at the time point when S₁ is measured; i.e., at the time point when the ratio of the movement signals of the magnetic particles becomes computable. For example, after S₀ and S₁ are measured: i.e., after the ratio of the movement signals of the magnetic particles becomes computable, the apparatus of the present disclosure may temporarily store the measured signals in a memory and then, after a given period of time, compute the ratio of the movement signals of the magnetic particles.

The phrase “period for computing the ratio of the movement signals of the magnetic particles” used herein refers to the period during which the ratio of the movement signals of the magnetic particles is computed. That is, the term refers to the time interval between the time point for computing the first ratio of the movement signals of the magnetic particles and the time point for computing the second ratio of the movement signals of the magnetic particles. Provided that the time point for monitoring the movement signal of the magnetic particles is mm₀, mm₁, mm₂, mm₃, mm₄ . . . , the time point for computing the ratio of the movement signals of the magnetic particles is mr₁, mr₂, mr₃, mr₄ . . . , and mm₁=mr₁, mm₂=mr₂, mm₃=mr₃, mm₄=mr₄ . . . , for example, the period for computing the ratio of the movement signals of the magnetic particles can be denoted as (mr₂−mr₁), (mr₃−mr₂), (mr₄−mr₃) . . . . In the figures, the period for computing the ratio of the movements signal of the magnetic particles may be indicated by an outlined thick arrow. In some embodiments, the period for computing the ratio of the movement signals of the magnetic particles may be constant. In other embodiments, the period for computing the ratio of the movement signals of the magnetic particles may be altered. In some embodiments, the period for computing the ratio of the movement signals of the magnetic particles can be selected from 0.1 seconds to 2 seconds.

In the method of the present disclosure, the period for monitoring the movement signal of the magnetic particles may be the same with or different from the period for computing the ratio of the movement signals of the magnetic particles. In some embodiments, for example, the period for monitoring the movement signal of the magnetic particles and the period for computing the ratio of the movement signals of the magnetic particles can both be 0.5 seconds, although the present disclosure is not limited thereto. In other embodiments, for example, the period for monitoring the movement signal of the magnetic particles may be set as 0.1 seconds, and the period for computing the ratio of the movement signals of the magnetic particles may be set as 0.5 seconds, although the present disclosure is not limited thereto.

In the present description, the time interval used to compute the ratio of the movement signals of the magnetic particles, when a signal rate (S₂/S₁) is to be computed, is the time interval from the time point at which S₁ is monitored to the time point at which S₂ is monitored. For example, let the time point for monitoring the movement signal of the magnetic particles be mm₀, mm₁, mm₂, mm₃, mm₄ . . . , the movement signal of the magnetic particles to be monitored at mm₀ be S₀, the movement signal of the magnetic particles to be monitored at mm₁ be S₁, the movement signal of the magnetic particles to be monitored at mm₂ be S₂, the movement signal of the magnetic particles to be monitored at mm₃ be S₃, the movement signal of the magnetic particles to be monitored at mm₄ be S₄ . . . , the time point for computing the ratio of the movement signals of the magnetic particles be mr₁, mr₂, mr₃, mr₄ . . . , mm₁=mr₁, mm₂=mr₂, mm₃=mr₃, mm₄=mr₄, the signal ratio computed at mr₁ be S₁/S₀, the signal ratio computed at mr₂, be S₂/S₁, the signal rate computed at mr₃ be S₃/S₂, and the signal ratio computed at mr₄ be S₄/S₃. Then, the time interval used to compute the ratio of the movement signals of the magnetic particles would be (mm₁−mm₀), (mm₂−mm₁), (mm₃−mm₂), (mm₄−mm₃) . . . . In the figures, the time interval used to compute the ratio of the movement signals of the magnetic particles may be indicated by a solid thick arrow. Incidentally, another signal not necessarily used for computing the signal ratio (S₁/S₀), may be present between S₀ and S₁. In other words, in some embodiments, all the measurement points (i.e., measured signals) need not necessarily be used for computing the signal ratio. In the method of the present disclosure, a constant time interval is employed for computing the ratio of the movement signals of the magnetic particles. That is, with reference to the example above, such time interval can be indicated as (mm₁−mm₀)=(mm₂−mm₁)=(mm₃−mm₂)=(mm₄−mm₃) . . . . In some embodiments, the time interval used to compute the ratio of the movement signals of the magnetic particles may be a constant time interval selected from between 0.1 seconds and 2 seconds, such as a time interval of 0.5 seconds, 1 second, 1.5 seconds, or 2 seconds, and may preferably be a time interval of 1 second.

An example of a reagent for fibrinogen determination used in the present disclosure is a fibrinogen measurement dry reagent containing highly active thrombin or a highly active thrombin-like protein, magnetic particles, a heparin neutralizer, a fibrin monomer polymerization inhibitor, a calcium salt, an amino acid or salt thereof, or a saccharide.

As an exemplary method for preparing the fibrinogen measurement dry reagent of the present disclosure, a buffer containing a fibrin monomer polymerization inhibitor, an amino acid or salt thereof, or a saccharide may be first prepared, highly active thrombin or a highly active thrombin-like protein may be dissolved in the buffer, magnetic particles may be added to the solution to prepare a final solution, a given amount of the final solution may be dispensed onto a reaction slide, and the solution may be frozen and lyophilized. The buffer may further contain a heparin neutralizer and/or a defoaming agent.

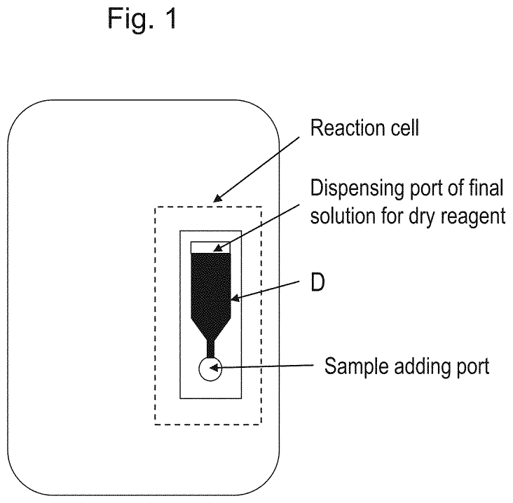

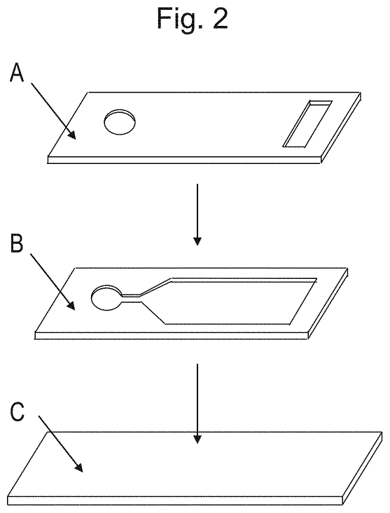

The reaction slide used in the method for preparation is not particularly limited, provided that with the reaction slide, an increase in viscosity in the fibrinogen measurement dry reagent at the time of fibrinogen measurement can be optically monitored as an attenuation in the movement signal of the magnetic particles. Examples include a reaction slide shown in FIGS. 1 and 2. FIG. 1 shows atop view of the reaction slide. In FIG. 1, an area surrounded by a dotted line is a reaction cell composed of a dispensing port for the final solution for preparing the fibrinogen measurement dry reagent and a sample adding port. FIG. 2 shows the structure of a reaction cell in detail. The reaction cell is constructed by first applying a transparent polyester plate B to a white polyester plate C and then applying a transparent polyester plate A to the transparent polyester plate B. First, a surfactant solution is introduced into the reaction cell through the dispensing port shown in FIG. 1 and then suction-removed to hydrophilize region D. Then, the final solution for the fibrinogen measurement dry reagent is injected into the reaction cell through the dispensing port to fill region D with the final solution. When this type of reaction slide is used, in general, 20 to 30 μl of the final solution for the fibrinogen measurement dry reagent can be dispensed onto the same. For methods for fibrinogen determination using magnetic particles such as this, see, for example, Patent Literature 2. The entire contents disclosed therein are incorporated herein by reference.

The reaction slide as shown in FIG. 1 may be referred to as a “dry reagent card” herein. In some embodiments, specifically, the fibrinogen measurement dry reagent according to the present disclosure can be applied to a dry reagent card.

In some embodiments, the dry reagent layer of the fibrinogen measurement dry reagent according to the present disclosure may preferably be (i) dissolved immediately after the sample is added dropwise thereto. In some embodiments, the dry reagent layer of the fibrinogen measurement dry reagent according to the present disclosure preferably (ii) shows no differences or substantially no difference in the dissolving rate among reagents. In some embodiments, with regard to the dry reagent layer of the fibrinogen measurement dry reagent according to the present disclosure, the dry reagent layer preferably (iii) is impact resistant (has impact resistance). In some embodiments, with regard to the fibrinogen measurement dry reagent according to the present disclosure, the dry reagent layer preferably (iv) is uniform. In some embodiments, with regard to the fibrinogen measurement dry reagent according to the present disclosure, preferably (v) the substance that is added to satisfy the conditions (i) to (iv) above imposes no influence or substantially no influence on the reaction. In some embodiments, the fibrinogen measurement dry reagent according to the present disclosure satisfies all of the conditions (i) to (v) above.

Unless indicated otherwise, contents of components of the fibrinogen measurement dry reagent described below indicate the weight and activity per 1 ml of the final solution to be dispensed onto the reaction slide shown in FIGS. 1 and 2.

In some embodiments, the fibrinogen measurement dry reagent according to the present disclosure comprises:

(i) thrombin or a protein having thrombin activity;

(ii) magnetic particles:

(iii) a fibrin monomer polymerization inhibitor:

(iv) a calcium salt;

(v) a dry reagent layer solubility improving agent;

(vi) a dry reagent layer reinforcing material: and

(vii) a pH buffer

as essential components. In other embodiments, the fibrinogen measurement dry reagent according to the present disclosure may further comprise, as optional components, a heparin neutralizer and/or a defoaming agent. In some embodiments, the fibrinogen measurement dry reagent according to the present disclosure is for use in measuring an undiluted plasma or whole blood sample.

The phrase “undiluted whole blood” used herein refers to whole blood, which is not subjected to any dilution procedure, such as the addition of a dilution buffer, with regard to the whole blood sample after blood sampling. As such, even if the blood is diluted with a citrate solution or other substances contained in the blood-sampling tube at the time of blood sampling (such blood is generally referred to as “citrated whole blood”) so long as the whole blood sample is not subjected to any specific dilution procedure after blood sampling, such blood is within the scope of “undiluted whole blood” as used herein. As such, undiluted whole blood encompasses citrated whole blood and heparinized whole blood that are not subjected to any dilution procedure. The phrase “undiluted plasma” used herein refers to a supernatant obtained by centrifugation of undiluted whole blood, and such plasma is not subjected to a dilution procedure, such as the addition of a dilution buffer. As such, the undiluted plasma encompasses citrated plasma and heparinized plasma that are not subjected to a dilution procedure. Incidentally, the phrase “non-diluted” is synonymous with the term “undiluted” in the present disclosure.

In some embodiments, the fibrinogen measurement dry reagent according to the present disclosure comprises thrombin or a protein having thrombin activity. A protein having thrombin activity may be referred to as a “thrombin-like protein” herein. The phrase “thrombin activity” used herein refers to activity capable of enhancing both the reactions: (i) conversion of fibrinogen into a fibrin monomer; and (ii) activation of factor XIII into factor XIIIa in the presence of a calcium ion. A protein having such activity is referred to as a protein having thrombin activity. It should be noted, however, that a single protein need not necessarily enhance both the reactions (i) and (ii) above. In other words, in certain embodiments, a mixture of (i) a first protein having thrombin activity of enhancing the conversion of fibrinogen into a fibrin monomer and (ii) a second protein having thrombin activity of enhancing the activation of factor XIII into factor XIIIa can be used. An example of the first protein is snake venom thrombin-like enzymes. The second protein may be a protein having activity of specifically cleaving a site between arginine 37 and glycine 38 from the N terminus of the factor XIII A subunit. Examples of thrombin or a protein having thrombin activity include, but are not limited to, bovine thrombin, human thrombin, and recombinants thereof. In some embodiments, thrombin or a protein having thrombin activity may be bovine thrombin. Bovine thrombin that is widely commercialized and readily available in the form of a lyophilized product may be used. Examples of thrombin or a protein having thrombin activity include, but are not limited to, a combination of snake venom thrombin-like enzymes and a protein having activity of specifically cleaving a site between arginine 37 and glycine 38 from the N terminus of the factor XIII A subunit. While the activity of thrombin or a protein having thrombin activity to be incorporated into the fibrinogen measurement dry reagent according to the present disclosure is not particularly limited, for example, the bovine thrombin activity level may be selected from the range of 100 to 500 NIHU/1 ml of the final solution, with the range of 150 to 400 NIHU/1 ml of the final solution being preferable.

In some embodiments, the fibrinogen measurement dry reagent according to the present disclosure comprises magnetic particles. Any conventional magnetic particles may be used for the fibrinogen measurement dry reagent according to the present disclosure without limitation. Examples of magnetic particles include, but are not limited to, triiron tetraoxide particles, iron sesquioxide particles, iron particles, cobalt particles, nickel particles, and chromium oxide particles. In some embodiments, magnetic particles can be fine particles of triiron tetraoxide. That is, in certain embodiments, fine particles of triiron tetraoxide are preferably used from the perspective of the intensity of the movement signal of the magnetic particles. The particle diameter of the magnetic particles is not particularly limited, and the average particle diameter can be 0.05 to 5 μm, 0.1 to 3.0 μm, such as 0.25 to 0.5 μm, although not limited thereto. In some embodiments, the average particle diameter of the magnetic particles may be 0.1 to 3.0 μm. The phrase “average particle diameter” used herein refers to a particle diameter (D50) at a cumulative value of 50% in a particle size distribution by a laser diffraction scattering method, unless otherwise specified. The magnetic particle content in the fibrinogen measurement dry reagent according to the present disclosure is not particularly limited. For example, such content may preferably be 4 to 40 mg/l ml of the final solution.

In some embodiments, the fibrinogen measurement dry reagent according to the present disclosure may further contain, as an optional component, a heparin neutralizer. Any conventional heparin neutralizer may be used as the heparin neutralizer without limitation, and examples thereof include, but are not limited to, polybrene, protamine sulfate, and heparinase. In some embodiments, polybrene may preferably be used as the heparin neutralizer from the perspective of good storage stability and cost effectiveness. The amount of a heparin neutralizer to be incorporated into a fibrinogen measurement dry reagent is not particularly limited and may be appropriately determined. When poly brene is used as a heparin neutralizer in an embodiment, the amount of polybrene to be incorporated into the fibrinogen measurement dry reagent may preferably be, for example, 50 to 300 μg/l ml of the final solution.

The fibrinogen measurement dry reagent according to the present disclosure comprises a fibrin monomer polymerization inhibitor. Any conventional inhibitor of fibrin monomer polymerization may be used (contained) in the fibrinogen measurement dry reagent according to the present disclosure without particular limitation. Examples of inhibitors of fibrin monomer polymerization include, but are not limited to, GPRP (glycine-proline-arginine-proline) peptide and derivatives thereof, such as GPRP-amide, and GHRP (glycine-histidine-arginine-proline) peptide and derivatives thereof, such as GHRP-amide. In other embodiments, the fibrin monomer polymerization inhibitor can be GPRPA (glycine-proline-arginine-proline-alanine) peptide and derivatives thereof, such as GPRPA-amide. In some embodiments, the fibrin monomer polymerization inhibitor may preferably be GPRP peptide and derivatives thereof from the perspective of affinity to fibrinogen. Such peptide is an analog of knob ‘A’ which is exposed when thrombin reacts with fibrinogen and fibrinopeptide A becomes released from the α chain of fibrinogen. When such peptide binds to hole ‘a’ that is present in the γ chain instead of knob ‘A,’ the same inhibits fibrin monomer polymerization (John WW: Mechanisms of fibrin polymerization and Clinical implications, Blood, 121 (10), 1712-1719, 2013).

The amount of a fibrin monomer polymerization inhibitor to be incorporated into a fibrinogen measurement dry reagent may appropriately be determined without particular limitation. When GPRP-amide is used as the fibrin monomer polymerization inhibitor, the amount of the GPRP-amide to be incorporated into the fibrinogen measurement dry reagent according to the present disclosure may preferably be 100 to 300 μg/1 ml of the final solution.

In some embodiments, the fibrinogen measurement dry reagent according to the present disclosure comprises a calcium salt. Any conventional calcium salt may be used for the dry reagent without limitation. Examples of inorganic acid calcium salts include calcium chloride, calcium nitrite, calcium sulfate, and calcium carbonate and the like. Examples of organic acid calcium salts include calcium lactate and calcium tartrate and the like. In some embodiments, calcium chloride is preferable as the calcium salt. The amount of calcium salt to be incorporated into a fibrinogen measurement dry reagent may appropriately be determined without particular limitation. When a calcium chloride dihydrate is used as the calcium salt, the amount of a calcium chloride dihydrate to be incorporated into the fibrinogen measurement dry reagent of the present disclosure may preferably be 0.2 to 2 μg/1 ml of the final solution.

In some embodiments, the fibrinogen measurement dry reagent according to the present disclosure comprises a dry reagent layer solubility improving agent (an agent for improving solubility of the dry reagent layer). Examples of the dry reagent layer solubility improving agent include an amino acid or salt thereof and a saccharide. The amino acid or salt thereof or a saccharide used herein may be any of a neutral amino acid or salt thereof, an acidic amino acid or salt thereof, a basic amino acid or salt thereof, a monosaccharide, and a polysaccharide. Examples of representative acidic amino acids or salts thereof include glutamic acid, sodium glutamate, aspartic acid, and sodium aspartate and the like. Examples of representative neutral amino acids or salts thereof include glycine, glycine hydrochloride, and alanine and the like. Examples of representative basic amino acids or salts thereof include lysine, lysine hydrochloride, and arginine and the like. Examples of monosaccharides include glucose and fructose and the like. Examples of polysaccharides include sucrose, lactose, and dextrin and the like. Among these substances, glycine is the most preferable from the perspective of good solubility of the reagent when a sample is added to the fibrinogen measurement dry reagent, good reproducibility of the motor signals of magnetic particles, and good impact resistance. In some embodiments, specifically, the dry reagent layer solubility improving agent used in the present disclosure may be glycine.

The amount of the dry reagent layer solubility improving agent to be incorporated into the fibrinogen measurement dry reagent used in the present disclosure, such as the amount of an amino acid or salt thereof or a saccharide, may appropriately be determined without particular limitation. When glycine is used as the dry reagent layer solubility improving agent, in some embodiments, the amount of glycine to be incorporated into the fibrinogen measurement dry reagent of the present disclosure may be 1.5% by weight or more, 1.6% by weight or more, 1.7% by weight or more, 1.8% by weight or more, 1.9% by weight or more, 2.0% by weight or more, 2.1% by weight or more, 2.2% by weight or more, 2.3% by weight or more, 2.4% by weight or more, 2.5% by weight or more, 2.6% by weight or more, 2.7% by weight or more, 2.8% by weight or more, 2.9% by weight or more, 3.0% by weight or more, 3.1% by weight or more, 3.2% by weight or more, 3.3% by weight or more, 3.4% by weight or more, 3.5% by weight or more, 3.6% by weight or more, 3.7% by weight or more, 3.8% by weight or more, 3.9% by weight or more, 4.0% by weight or more, 4.1% by weight or more, 4.2% by weight or more, 4.3% by weight or more, 4.4% by weight or more, 4.5% by weight or more, 4.6% by weight or more, 4.7% by weight or more, 4.8% by weight or more, or 4.9% by weight or more, such as 5.0% by weight. When glycine is used as the dry reagent layer solubility improving agent, in some embodiments, the amount of glycine to be incorporated into the fibrinogen measurement dry reagent of the present disclosure may be 5.0% by weight or less, 4.9% by weight or less, 4.8% by weight or less, 4.7% by weight or less, 4.6% by weight or less, 4.5% by weight or less, 4.4% by weight or less, 4.3% by weight or less, 4.2% by weight or less, 4.1% by weight or less, 4.0% by weight or less, 3.9% by weight or less, 3.8% by weight or less, 3.7% by weight or less, 3.6% by weight or less, 3.5% by weight or less, 3.4% by weight or less, 3.3% by weight or less, 3.2% by weight or less, 3.1% by weight or less, 3.0% by weight or less, 2.9% by weight or less, 2.8% by weight or less, 2.7% by weight or less, 2.6% by weight or less, 2.5% by weight or less, 2.4% by weight or less, 2.3% by weight or less, 2.2% by weight or less, 2.1% by weight or less, 2.0% by weight or less, 1.9% by weight or less, 1.8% by weight or less, 1.7% by weight or less, or 1.6% by weight or less, such as 1.5% by weight. In the present disclosure, the amount of glycine to be incorporated into the fibrinogen measurement dry reagent of the present disclosure encompasses any combination of minimal amount and maximal amount wherein the minimal amount and maximal amount are set to be any of the minimal amounts and the maximal amounts mentioned above. For example, in some embodiments, the amount of glycine to be incorporated into the fibrinogen measurement dry reagent of the present disclosure may be set as 1.5% to 5.0% by weight, 2.0% to 5.0% by weight, 2.5% to 5.0% by weight, 3.0% to 5.0% by weight, 3.5% to 5.0% by weight, 4.0% to 5.0% by weight, 4.5% to 5.0% by weight, 1.5% to 4.5% by weight, 2.0% to 4.5% by weight, 2.5% to 4.5% by weight, 3.0% to 4.5% by weight, 3.5% to 4.5% by weight, 4.0% to 4.5% by weight, 1.5% to 4.0% by weight, 2.0% to 4.0% by weight, 2.5% to 4.0% by weight, 3.0% to 4.0% by weight, 3.5% to 4.0% by weight, 1.5% to 3.5% by weight, 2.0% to 3.5% by weight, 2.5% to 3.5% by weight, 3.0% to 3.5% by weight, 1.5% to 3.0% by weight, 2.0% to 3.0% by weight, 2.5% to 3.0% by weight, 1.5% to 2.5% by weight, 2.0% to 2.5% by weight, or 1.5% to 2.0% by weight. In some embodiments, when glycine is used as the dry reagent layer solubility improving agent, the amount of glycine to be incorporated into the fibrinogen measurement dry reagent of the present disclosure may preferably be 1.5% to 4.0% by weight. In other embodiments, when glycine is used as the dry reagent layer solubility improving agent, the amount of glycine to be incorporated into the fibrinogen measurement dry reagent of the present disclosure may preferably be 2.0% to 3.0% by weight. When glycine is used as the dry reagent layer solubility improving agent for measuring an undiluted plasma sample, the amount of glycine to be incorporated into the fibrinogen measurement dry reagent of the present disclosure may be within the range mentioned above, such as 1.5% to 4.0% by weight. When glycine is used as the dry reagent layer solubility improving agent for measuring an undiluted whole blood sample, the amount of glycine to be incorporated into the fibrinogen measurement dry reagent of the present disclosure may be within the range mentioned above, such as 1.5% by weight or more. When glycine is used as the dry reagent layer solubility improving agent for measuring an undiluted whole blood sample, for example, the amount of glycine to be incorporated into the fibrinogen measurement dry reagent of the present disclosure may be 1.5% to 5.0% by weight, or 1.5% to 4.5% by weight, such as 1.5% to 4.0% by weight. When enabling measurement of both an undiluted plasma sample and an undiluted whole blood sample, when glycine is used as the dry reagent layer solubility improving agent, the amount of glycine to be incorporated into the fibrinogen measurement dry reagent of the present disclosure may be any combination of these various ranges. It should be noted that the unit “% by weight” used herein indicates the concentration in the final solution: i.e., the final concentration, unless otherwise specified.

In some embodiments, the fibrinogen measurement dry reagent according to the present disclosure comprises a buffer. Prior to lyophilization, a buffer supplemented with a protein having thrombin activity, magnetic particles, a heparin neutralizer, a fibrin monomer polymerization inhibitor, a calcium salt, and a dry reagent layer solubility improving agent is not particularly limited, provided that buffering actions at pH 6.0 to 8.0. In some embodiments, a buffer may be a buffer capable of adjusting the pH level of the reagent to a pH of 6.0 to 8.0, such as about pH 7.35 or about pH 7.5. Examples of buffers include 40 mM HEPES-NaOH buffer (pH=7.35) and 40 mM Tris-HCl buffer (pH=7.5) as preferable buffers.

In some embodiments, the fibrinogen measurement dry reagent according to the present disclosure comprises a dry reagent layer reinforcing material (a material for reinforcing the dry reagent layer). Examples of the dry reagent layer reinforcing material include, but are not limited to, bovine serum albumin and human serum albumin. When bovine serum albumin is used as the dry reagent layer reinforcing material, the amount of dry reagent layer reinforcing material to be incorporated into the dry reagent may preferably be in a range of 0.6 to 2.0 mg/I ml of the final solution.

In some embodiments, the fibrinogen measurement dry reagent according to the present disclosure may comprise, as an optional component, a defoaming agent. Examples of the defoaming agent include, but are not limited to, sorbitan monolaurate, a silicone-based defoaming agent, and a polypropylene glycol-based defoaming agent. When sorbitan monolaurate is used as the defoaming agent, the amount of defoaming agent to be incorporated into the dry reagent may preferably be in a range of about 0.001% to about 0.010% by weight.

The method of drying the buffer containing the components described above is preferably lyophilization, from the perspective of solubility of a fibrinogen measurement dry reagent, the movement signal intensity of magnetic particles, and reproducibility. When a buffer is air-dried, solubility of the reagents is poor, and the movement signals of magnetic particles are weak and, therefore, it is difficult to detect the end point. Further, when a buffer is air-dried, the clotting time determined based on the end point (even if detected) may not necessarily correspond to the fibrinogen concentration.

The method of freezing and lyophilization are not particularly limited. Common techniques of freezing can be employed and, for example, a final solution for the fibrinogen measurement dry reagent can be dispensed onto a reaction slide through the dispensing port shown in FIG. 1 and then the reaction slide can be stored and frozen in a freezer maintained at −40° C. or lower for one whole day and night, the reaction slide can be mounted on a lyophilizer in which the shelf temperature is set at −40° C. or lower and stored and frozen therein for one whole day and night, or the reaction slide can be frozen instantly with liquid nitrogen. In addition, the technique for lyophilizing the frozen reaction slide is not particularly limited. To exemplify the lyophilizing method, the lyophilizing method includes a method in which the temperature of the frozen reaction slide may be linearly raised from −30° C. to −20° C. over a period of 24 hours in vacuum, the temperature thereof may be linearly raised from −20° C. to 30° C. over a period of 20 hours, lastly the temperature may be maintained at 30° C. for 3 hours, and then dry air may be applied to release the vacuum.

With regard to the lyophilized reagent for fibrinogen measurement above, it is preferably to immediately seal the same with an aluminum film in a dehumidified environment. While the dehumidified environment is not particularly limited, an environment in which temperature is at room temperature of 22° C. to 27° C. and relative humidity of 35% or lower is preferable. Further, while specifications of the aluminum film are not particularly limited, a preferable aluminum film includes a 5-layer structure aluminum film (thickness: 86 μm) comprising a polyester film (thickness: 12 μm), polyethylene resin (thickness: 15 μm), an aluminum foil (thickness: 9 μm), a polyethylene resin (thickness: 20 μm), and a polyethylene film (thickness: 30 μm) adhered with an AC coating agent. The entire fibrinogen measurement dry reagent is wrapped with the aluminum foil and sealed via heat adhesion. It is preferable to refrigerate the fibrinogen measurement dry reagent in a sealed state before using the same for fibrinogen measurement.

In a particular embodiment, fibrinogen determination involving the use of the fibrinogen measurement dry reagent according to the present disclosure may be performed by adding an sample to the reagent to dissolve the reagent and using an apparatus that applies a combination of an oscillating magnetic field and a static permanent magnetic field to allow the magnetic particles contained in the reagent to move, detects the movement signal of the magnetic particles as the amount of change in the scattered light, detects the clotting point based on the change with the elapse of time and computes the clotting time as the time from the starting point (the starting point of the coagulation reaction) to the clotting point. The obtained clotting time is correlated with the fibrinogen concentration in the sample.

With regard to the determination method according to the present disclosure, the given range of the ratio of the movement signals of the magnetic particles is not particularly limited. For example, the given range of the ratio of the movement signals of the magnetic particles can be in a range from 1.0±0.05 to 1.0±0.2, such as 1.0±0.2, 1.0±0.19, 1.0±0.18, 1.0±0.17, 1.0±0.16, 1.0±0.15, 1.0±0.14, 1.0±0.13, 1.0±0.12, 1.0±0.11, 1.0±0.1, 1.0±0.09, 1.0±0.08, 1.0±0.07, 1.0±0.06, or 1.0±0.05. In some embodiments, the given range of the ratio of the movement signals of the magnetic particles may preferably be 1.0±0.05 to 1.0±0.15 and particularly preferably 1.0±0.1 from the perspective of good reproducibility of the clotting time. In other words, the given range of the ratio of the movement signals of the magnetic particles may be a range of 0.8 to 1.2, a range of 0.81 to 1.19, a range of 0.82 to 1.18, a range of 0.83 to 1.17, a range of 0.84 to 1.16, a range of 0.85 to 1.15, a range of 0.86 to 1.14, a range of 0.87 to 1.13, a range of 0.88 to 1.12, a range of 0.89 to 1.11, a range of 0.9 to 1.1, a range of 0.91 to 1.09, a range of 0.92 to 1.08, a range of 0.93 to 1.07, a range of 0.94 to 1.06, or a range of 0.95 to 1.05 and the like. A range of 0.9 to 1.1 is particularly preferable from the perspective of good reproducibility of the clotting time.

With regard to the determination method according to the present disclosure, the time (the interval) during which the ratio of the movement signals of the magnetic particles is maintained within a given range is not particularly limited. For example, the time (the interval) during which the ratio of the movement signals of the magnetic particles is maintained within a given range can be 1 to 5 seconds, 1 to 4 seconds, 1 to 3 seconds, 5 seconds, 4.5 seconds, 4 seconds, 3.5 seconds, 3 seconds, 2.5 seconds, 2 seconds, 1.5 seconds, or 1 second, although the time (the interval) is not limited thereto. In some embodiments, the time (the interval) during which the ratio of the movement signals of the magnetic particles is maintained within a given range may preferably be 1 to 3 seconds, and more preferably 1.5 seconds, from the perspective of good reproducibility of the clotting time.

With regard to the determination method according to the present disclosure, the starting point is any point within an interval during which a plurality of ratios of the movement signals of the magnetic particles are monitored at a given time interval and the ratio is maintained within a given range for a given period of time. The ratio of the movement signals of the magnetic particles a given time interval can be monitored continuously or intermittently. In a particular embodiment, the first point within the time period during which the ratio of the movement signals of the magnetic particles is maintained within a given range for a given period of time can be designated as the starting point. In other embodiments, the starting point can be a point other than the first point within the time period during which the rate is maintained within a given range for a given period of time, such as the second, the third, or the fourth point within the time period during which the rate is maintained within a given range for a given period of time. Such embodiments are substantially within the scope of the present disclosure. Incidentally, in order to avoid the initial variability of the signals after the addition of the sample, the starting point is defined in the method of the present disclosure for the convenience of description and, for example, such point is described as the point of measurement time 0 (sec) in the tables. However, this does not mean that the coagulation reaction is not initiated at all before the point of measurement time 0 (sec).

With regard to the determination method according to the present disclosure, unless otherwise specified the peak value is the peak value of the movement signal of the magnetic particles observed at or after the starting point and this is the maximal movement signal of the magnetic particles among the signals of the magnetic particles at or after the starting point. This peak value according to the present disclosure is different from the peak value according to conventional techniques. That is, according to the method described in JP H06-141895 A (JP Patent No. 2980468), the maximal signal among all the measured signals was simply designated as the peak value. However, when the present inventors applied the dry reagent described in Example 1 to the determination method according to JP H06-141895 A (JP Patent No. 2980468), the movement signal of the magnetic particles varied to a significant extent in the initial measurement stage after addition of the sample. When the maximal signal among all the measured signals was designated as the peak value, there were instances where fibrinogen determination could not be performed correctly. Therefore, in a particular embodiment of the present disclosure, the starting point is defined, and the peak value of the movement signal of the magnetic particles at or after the starting point is correctly defined, thereby determining fibrinogen more accurately with regard to undiluted samples.

With regard to the determination method according to the present disclosure, the end point is an arbitrary point among the points where the signal is attenuated by 5% to 50% from the peak value of the movement signal of the magnetic particles at or after the starting point defined in the manner described above. For example, when the peak value of the movement signal of the magnetic particles at or after the starting point is designated as 100%, a point at which the movement signal of the magnetic particles is equivalent to 70% of the peak value of the movement signal is referred to as a point attenuated by 30% herein (point attenuated by 30% from the peak value). For example, the end point can be a point attenuated by 5% to 50%, a point attenuated by 10% to 45%, a point attenuated by 15% to 40%, a point attenuated by 20% to 35%, a point attenuated by 20% to 30%, such as a point attenuated by 20%, a point attenuated by 25%, or a point attenuated by 30%, relative to the peak value of the movement signal of the magnetic particles at or after the starting point, although the end point is not limited thereto. A point attenuated by 30% from the peak value of the movement signal of the magnetic particles is particularly preferable from the perspective of good reproducibility of the clotting time. In some embodiments, the end point can be defined in depending on the type of sample; i.e., whether the measurement blood sample is an undiluted whole blood sample or undiluted plasma sample. That is, when an undiluted whole blood sample is to be measured, the end point can be a point attenuated by 20% relative to the peak value of the movement signal of the magnetic particles at or after the starting point. For example, when the blood sample to be measured is an undiluted plasma sample, the end point can be a point attenuated by 30% from the peak value of the movement signal of the magnetic particles at or after the starting point. Each end point applied according to the different sample type can appropriately be selected from among the points attenuated by 5% to 50% from the peak value of the movement signal of the magnetic particles at or after the starting point. Incidentally, the phrase “the peak value of the movement signal of the magnetic particles at or after the starting point” used herein refers to the maximal signal (C) among the movement signals of the magnetic particles measured at or after the starting point, and this may include the starting point itself. In other words, if the movement signal of the magnetic particles at the starting point is the maximal signal among the movement signals of the magnetic particles measured at or after the starting point, then such movement signal is the peak value of the movement signals of the magnetic particles at or after the starting point.

The phrase “clotting time” used in the present disclosure refers to the time from the starting point to the end point. That is, with regard to the fibrinogen determination method according to the present disclosure, the clotting time is computed as the time from the starting point to the end point. The obtained clotting time is correlated with the fibrinogen concentration. Examples of an apparatus that can implement the fibrinogen determination method according to the present disclosure include CG02N (product name: commercialized by A&T Corporation), although apparatuses that can be used are not limited thereto.

CG02N is an apparatus suitable for a conventional fibrinogen determination method (JP H06-141895 A (JP Patent No. 2980468)). After a sample is added to the fibrinogen measurement dry reagent, a combination of an oscillating magnetic field and a static permanent magnetic field is applied at an interval of 0.5 seconds, and the movement signals of the magnetic particles are monitored at the same interval. In order to implement the fibrinogen determination method according to the present disclosure with such apparatus, in addition to the foregoing, in particular embodiments, for example, the ratio of the movement signals of the magnetic particles is continuously computed at an interval of 1 second, and the first point of the interval during which the ratio is maintained within a range of 1.0±0.1 for 1.5 seconds can be detected as the starting point. In this regard, a point attenuated by 5% to 50%, such as a point attenuated by 30%, relative to the peak value of the movement signal of the magnetic particles at or after the starting point may be designated as the end point, and the time from the starting point to the end point can be computed as the clotting time. It should be noted that this is merely one example and the present disclosure is not limited thereto.

A series of operation including such arithmetic processing may be carried out by controlling the apparatus with a program or software. The program or software may be integrated in the apparatus or recorded on an information recording medium. In one embodiment, the present disclosure provides a program or software for executing (implementing) the fibrinogen determination method. In one embodiment, the present disclosure provides an information recording medium comprising the program or software recorded thereon. In one embodiment, the present disclosure provides an apparatus for fibrinogen determination comprising a program or software for executing the fibrinogen determination method integrated therein or the information recording medium stored therein. In some embodiments, the apparatus for fibrinogen determination encompasses an apparatus comprising the program of the present disclosure integrated in the CG02N apparatus.

Table 1 shows an example of whole blood sample measurements performed by the fibrinogen determination method according to the present disclosure. In such method, monitoring of the movement signal of the magnetic particles is initiated immediately after the addition of the sample and monitoring is performed at an interval of 0.5 seconds. That is, the period for monitoring the movement signal of the magnetic particles is 0.5 seconds. The ratio of the movement signals of the magnetic particles is then continuously computed at an interval of 1 second. In other words, the time interval used to compute the ratio of the movement signals of the magnetic particles is 1 second. That is, the ratio of the movement signals of the magnetic particles is computed as follows: (the movement signal of the magnetic particles detected at the monitoring time of 1.0 second)/(the movement signal of the magnetic particles detected at the monitoring time of 0 seconds). (the movement signal of the magnetic particles detected at the monitoring time of 1.5 seconds)/(the movement signal of the magnetic particles detected at the monitoring time of 0.5 seconds), (the movement signal of the magnetic particles detected at the monitoring time of 2.0 seconds)/(the movement signal of the magnetic particles detected at the monitoring time of 1.0 second) . . . . The interval during which the rate is maintained within a range of 1.0±0.1 for 1.5 seconds is the period of the monitoring time of 5.0 to 6.5 seconds. The first point thereof is the monitoring time of 5.0 seconds, and this point can be thus designated as the starting point (the starting point of the coagulation reaction: the time point of measurement time 0 (sec)). The peak value of the movement signal of the magnetic particles at or after the starting point is 2726c detected at the monitoring time of 7.0 seconds. The movement signal of the magnetic particles that is lower by 30% than the peak value of the movement signal of the magnetic particles at or after the starting point is computed to be 1908c. That is, the end point is the point at which the movement signal of the magnetic particles is 1908c, and the clotting time is computed to be 20.1 seconds. Since the movement signal of the magnetic particles 1908c is a computed value, the corresponding monitoring time and ratios of the movement signals of the magnetic particles at an interval of 1 second are not shown in the table. That is, the clotting time determined by the method of the present disclosure need not necessarily be one of the actual measurement points (one of the actual monitoring time points).

TABLE 1

| Time point | | Ratio of movement | | |
|---|---|---|---|---|
| for | | signal of magnetic | | |
| monitoring | | particle at the time | | |
| movement | Movement | interval of 1 sec for | | |
| signal of | signal of | calculating the rate | | |
| the magnetic | magnetic | of movement signals | Start/ | Measurement |
| particles | particles | of the magnetic | peak/ | time |
| (sec) | (C) | particles (−) | end | (sec) |
| 0 | 1430 | — | | |
| 0.5 | 86 | — | | |
| 1.0 | 359 | 0.25 | | |
| 1.5 | 114 | 1.33 | | |
| 2.0 | 3722 | 10.37 | | |
| 2.5 | 4235 | 37.15 | | |
| 3.0 | 1841 | 0.49 | | |
| 3.5 | 3534 | 0.83 | | |
| 4.0 | 2890 | 1.57 | | |
| 4.5 | 2389 | 0.68 | | |
| 5.0 | 2673 | 0.92 | Start | 0 |
| 5.5 | 2581 | 1.08 | | 0.5 |
| 6.0 | 2682 | 1.00 | | 1.0 |
| 6.5 | 2651 | 1.03 | | 1.5 |
| 7.0 | 2726 | 1.02 | Peak | 2.0 |
| 7.5 | 2678 | 1.01 | | 2.5 |
| 8.0 | 2721 | 1.00 | | 3.0 |
| 8.5 | 2673 | 1.00 | | 3.5 |
| 9.0 | 2708 | 1.00 | | 4.0 |
| 9.5 | 2665 | 1.00 | | 4.5 |
| 10.0 | 2677 | 0.99 | | 5.5 |
| 10.5 | 2635 | 0.99 | | 6.0 |
| 11.0 | 2635 | 0.98 | | 6.5 |
| . | . | . | | . |
| . | . | . | | . |
| . | . | . | | . |
| 22.0 | 2031 | 0.98 | | 17.5 |
| 22.5 | 2007 | 0.98 | | 18.0 |
| 23.0 | 1980 | 0.97 | | 18.5 |
| 23.5 | 1957 | 0.98 | | 19.0 |
| 24.0 | 1940 | 0.98 | | 19.5 |
| 24.5 | 1913 | 0.98 | | 20.0 |
| — | 1908 | — | End | 20.1 |
| 25.0 | 1889 | 0.97 | | 20.5 |
| 25.5 | 1870 | 0.98 | | 21.0 |

Peak movement signal of magnetic particles after the starting point: 2726
Movement signal of magnetic particles attenuated by 30% from the peak movement signal of magnetic particles after the starting point: 1908
Clotting time = 20.1 sec

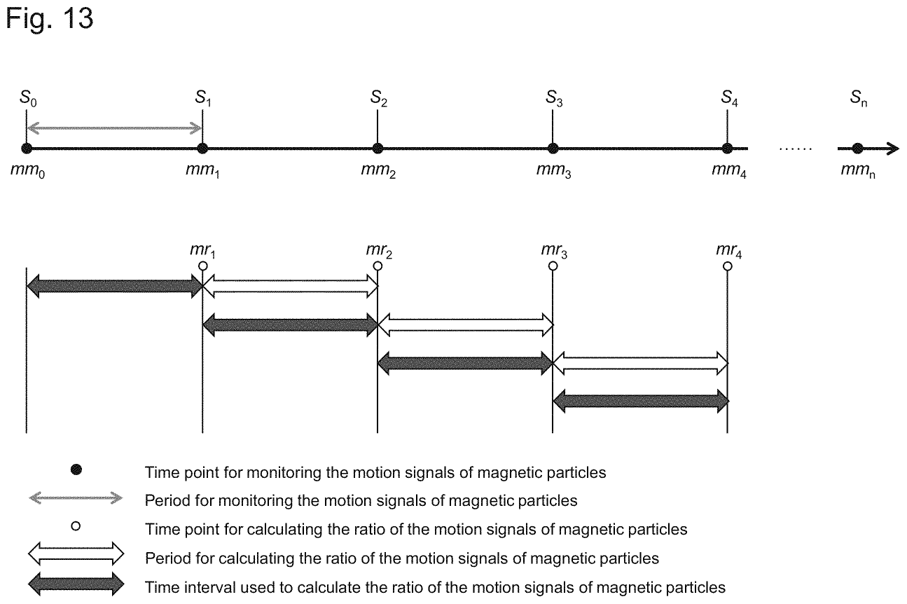

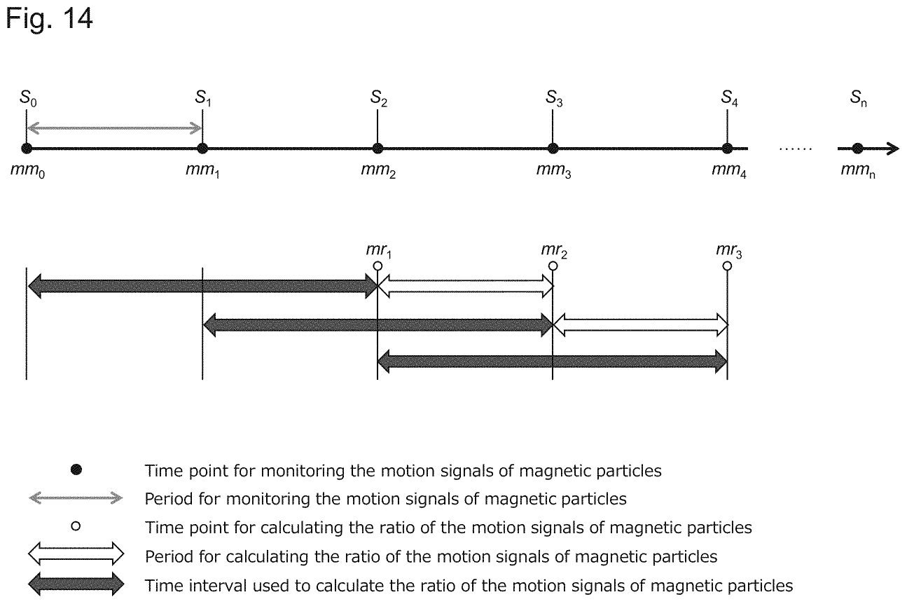

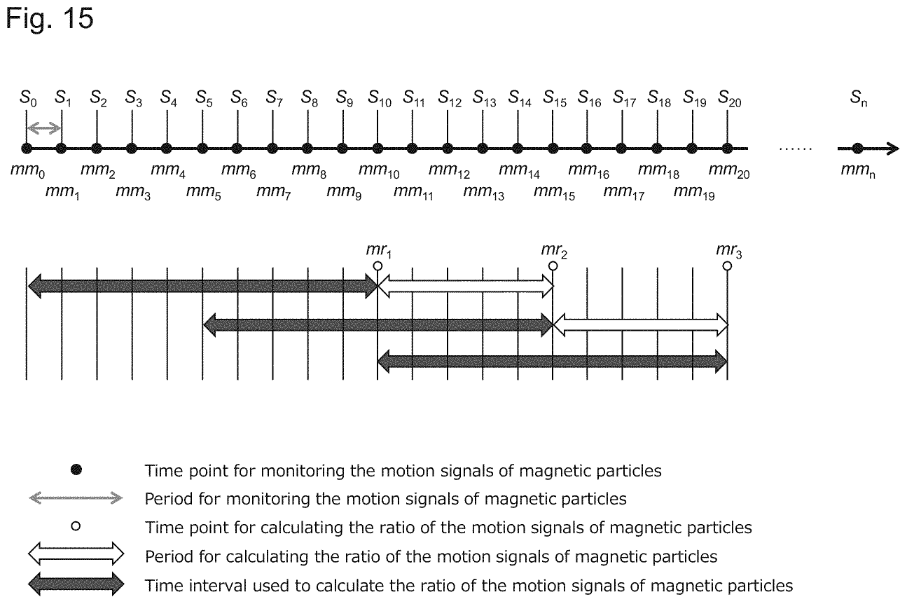

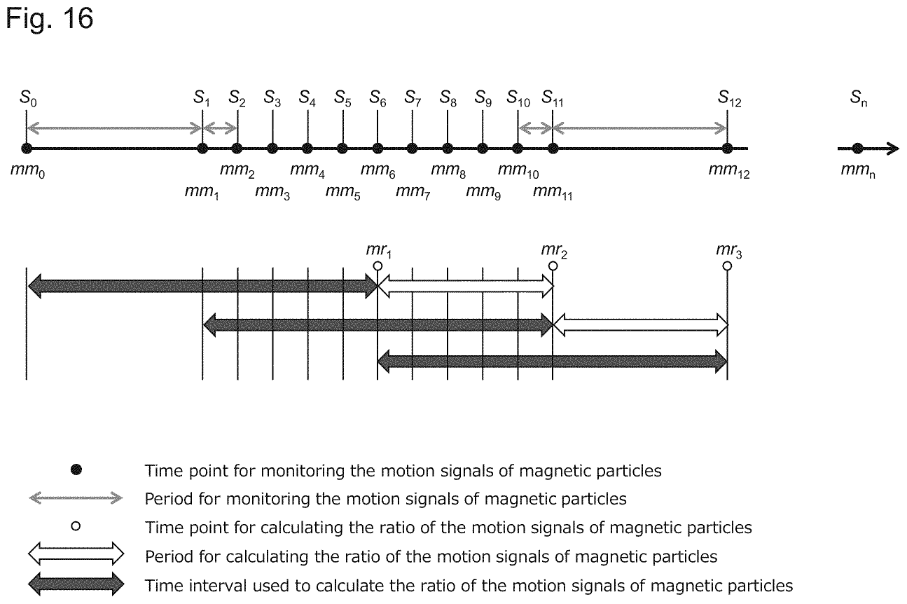

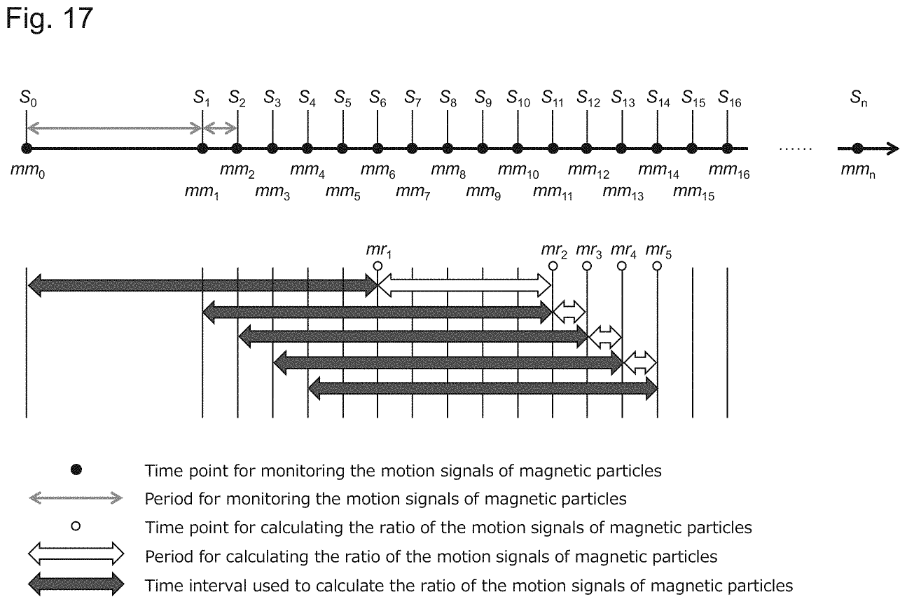

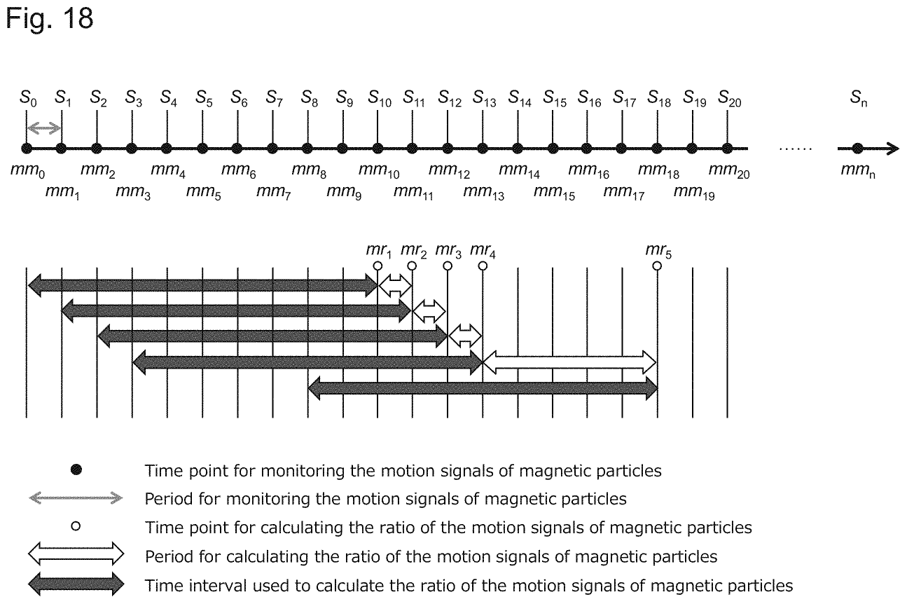

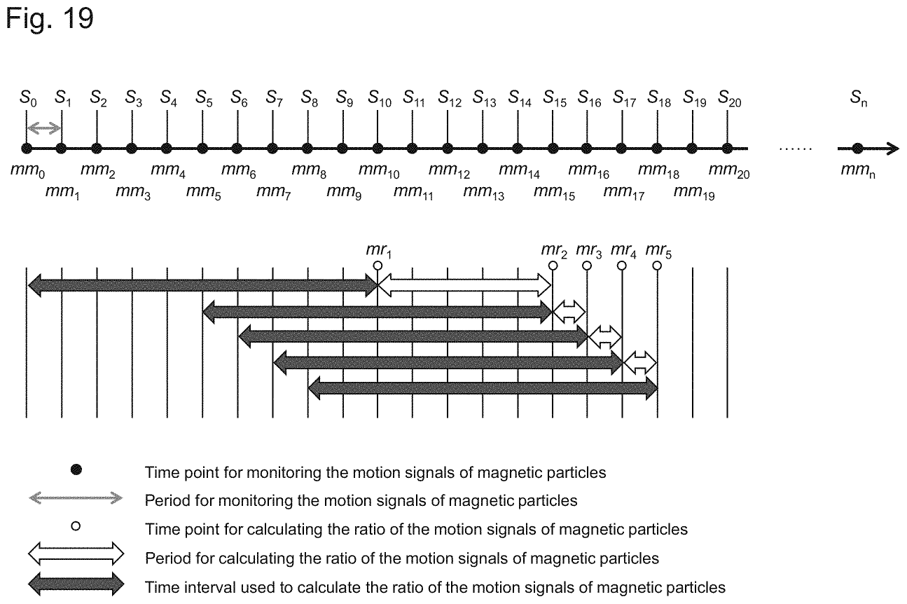

Incidentally, the fibrinogen determination method according to the present disclosure is not limited to the above. The period for monitoring the movement signal of the magnetic particles, the period for computing the ratio of the movement signals of the magnetic particles, and the time interval employed for computing the signal ratio of magnetic particles may all be the same (e.g., FIG. 13) or may be different (e.g., FIGS. 14 and 15). The period for monitoring the movement signal of the magnetic particles may be constant (e.g., FIGS. 13, 14, and 15) or may be altered (e.g., FIG. 16). The period for monitoring the movement signal of the magnetic particles and periods for computing the ratio of the movement signals of the magnetic particles may be constant (e.g., FIGS. 13, 14, and 15) or may be altered (e.g., FIG. 17). The ratio of the movement signals of the magnetic particles may be computed continuously (e.g., FIGS. 13 and 14) or intermittently (e.g., FIG. 15). Alternatively, the ratio of the movement signals may be computed continuously and then intermittently (e.g., FIG. 18) or the same may be computed intermittently and then continuously (e.g., FIG. 19). Various periods for monitoring the movement signal of the magnetic particles, various periods for computing the ratio of the movement signals of the magnetic particles, and various time intervals for computing the signal ratio of magnetic particles can be employed. However, it is preferable that the conditions to prepare a calibration curve and the conditions under which the sample is measured are the same conditions. Other various embodiments which become apparent from descriptions herein also encompassed by the present disclosure.

The fibrinogen determination method of a citrated plasma sample which utilizes said clotting time is not particularly limited. A representative example is as follows. First, 3 types of citrated plasma samples with known but different fibrinogen concentrations are measured by the method described above, the clotting times corresponding to the citrated plasma samples are obtained, and a calibration curve is prepared based thereon in advance. Subsequently, a citrated plasma sample is measured by the method described above, the clotting time is obtained, and the fibrinogen concentration in the citrated plasma sample is determined using the calibration curve prepared above. The calibration curve used in such method may preferably be a linear regression calibration curve with the Y axis indicating LN (fibrinogen concentration) and the X axis indicating LN (clotting time). The determined linear regression is a linear formula (Y=A×X+B), and the fibrinogen concentration in the citrated plasma sample is computed based on the slope of the linear formula (A) and the intercept (B) with the formula shown below.

Fibrinogen concentration in citrated plasma sample=e^{B}×(clotting time)^{A} [Formula 1]

In a particular embodiment, an example of an apparatus that can be used for fibrinogen determination involving the use of the fibrinogen measurement dry reagent according to the present disclosure includes the CG02N blood coagulation analyzer (A&T Corporation). This apparatus can be operated by designating the point attenuated by 30% relative to the peak value of the movement signal of the magnetic particles detected at or after the starting point (the starting point of the coagulation reaction) as the clotting point and designating the period from the starting point (the starting point of the coagulation reaction) to the clotting point as the clotting time. The ratio of the movement signals of the magnetic particles can be computed continuously at a given time interval and the starting point can be designated as the first point of the interval during which the ratio is maintained within a given range for a given period of time.

In general, the fibrinogen concentration in a sample is expressed as the fibrinogen concentration in citrated plasma. Since whole blood samples comprise blood cell components in addition to plasma components, it is necessary to take the hematocrit value of the sample into consideration when performing fibrinogen determination on whole blood samples. That is, when using a whole blood sample, it is necessary to subject the fibrinogen concentration converted from the clotting time determined by whole blood measurement to hematocrit correction in order to determine the fibrinogen concentration of the sample. In the case of citrated whole blood, it is necessary to add 9 volumes of whole blood to 1 volume of a sodium citrate solution and mix them with each other to obtain an assay sample (measurement sample). In contrast, in the case of a heparinized whole blood, an assay sample is obtained by adding whole blood to heparin sodium or heparin lithium powder and mixing them with each other. As such, the hematocrit correction formula adopted in the case of citrated whole blood is different from that adopted in the case of heparinized whole blood. In the case of citrated whole blood, specifically, the fibrinogen concentration in the sample is computed with the correction formula below.

Fibrinogen concentration in sample=fibrinogen concentration in citrated whole blood×(100/(100−hematocrit value×0.9)) [Formula 2]

When heparinized whole blood is used as the sample, the fibrinogen concentration in the sample is computed with the correction formula below.

Fibrinogen concentration in sample=fibrinogen concentration in heparinized whole blood×0.9×(100/(100−hematocrit value)) [Formula 3]

Incidentally, when citrated whole blood is used as the assay sample, and the hematocrit value is determined using citrated whole blood, the fibrinogen concentration in the sample is computed with the correction formula below.

Fibrinogen concentration in sample=fibrinogen concentration in citrated whole blood×(100/(100−hematocrit value)) [Formula 4]

Incidentally, if whole blood is filtered through a filter or a filter medium that does not substantially adsorb fibrinogen, plasma that is suitable for fibrinogen determination can be obtained in a simple manner without using a centrifuge. A plasma sample thus obtained may be applied to the present invention, thereby enabling accurate and simple fibrinogen concentration determination without the need to perform the five corrections using the correction formulae disclosed above.

The results of fibrinogen determination by the method of the present disclosure is extremely consistent with the results of fibrinogen determination by the conventional Clauss method. In addition, the method of the present disclosure yields good reproducibility, and reliable determination can be carried out even when using undiluted whole blood as the sample. Further, reliable determination can be carried out when using undiluted plasma as the sample.

According to the present disclosure, fibrinogen can be determined rapidly and accurately without the need for preparing reagents or carrying out diluting procedures on the sample. The present disclosure provides a fibrinogen measurement dry reagent that can be used in the perinatal period and perioperative period. In some embodiments, specifically, the fibrinogen measurement dry reagent according to the present disclosure is used for patients in the perinatal period. In other embodiments, the fibrinogen measurement dry reagent according to the present disclosure is used for patients in the perioperative period. The phrase “perinatal period” used herein refers to a period from the 22nd week of pregnancy to before 7 days from birth. This is a definition in accordance with the definition of “the perinatal period” of the International Classification of Diseases, Tenth Revision. Further, the phrase “perioperative period” used herein refers to a period including the 3 phases necessary for surgery; i.e., preoperative, intra-operative, and postoperative phases.

### EXAMPLES

The present invention has been described in general terms above. However, the present invention can be further understood with reference to the specific examples below. It should be noted that the examples presented here are provided solely for illustrative purposes and do not limit the scope of the present invention including those described in the claims.

### Example 1: Correlation Between Fibrinogen Concentration in Plasma and Clotting Time

A 40 mM HEPES buffer (pH 7.35) supplemented with 10 mM CaCl₂.2H₂O, 2.0 (wt/v) % glycine, 80 μg/ml polybrene, 1.2 mg/ml bovine serum albumin, 0.005 (wt/v) % sorbitan monolaurate, and 150 μg/ml GPRP-amide was added to a lyophilized bovine thrombin product (Oriental Yeast Co., Ltd.) and dissolved to obtain a reagent solution having 300 NIHU/ml of thrombin activity. To 35 ml of the reagent solution, 0.47 g of triiron tetraoxide (product name: AAT-03; average particle diameter: 0.35 μm; Toda Kogyo Corp.) was added and suspended to obtain a final solution. The final solution (25 μl) was dispensed onto the reaction slide shown in FIG. 1. The reaction slide was stored and frozen in a freezer maintained at −40° C. for one whole day and night. Subsequently, the frozen reaction slide was lyophilized. Lyophilization was performed under the conditions in which the temperature was linearly raised from −30° C. to −20° C. over a period of 24 hours in vacuum, the temperature was linearly raised from −20° C. to 30° C. over a period of 20 hours, the temperature was maintained at 30° C. for 3 hours, and then dry air was applied to release the vacuum. The lyophilized reagent was immediately sealed with an aluminum film in a dehumidified environment.

The method for examining the correlation between the fibrinogen concentration in plasma and the clotting time was carried out in the manner described below. First, human plasma containing 299 mg/dl of fibrinogen and fibrinogen-deficient plasma (Clinisys Associate) were used to prepare 6 serial dilution samples of human plasma from 48 to 299 mg/dl. Subsequently, the lyophilized reagent was mounted on the CG02N blood coagulation analyzer (A&T Corporation), 25 μl each of the serial dilution samples were added thereto, and the clotting time of each sample was determined, the data were plotted by setting the Y axis to LN (fibrinogen concentration) and the X axis to LN (clotting time), and whether or not linearity could be observed in the prepared chart was examined to inspect whether there was a correlation or not.

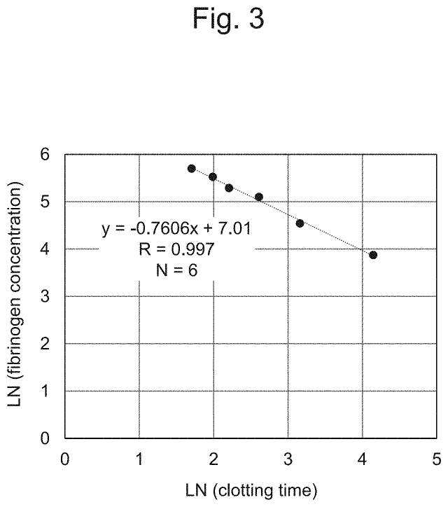

FIG. 3 shows the correlation between the fibrinogen concentration in plasma and the clotting time. As is apparent from FIG. 3, the clotting time was extremely well-correlated with the fibrinogen concentration in the sample.

### Example 2: Specificity and Reproducibility of Fibrinogen Concentration in Plasma

As the fibrinogen measurement dry reagent, the lyophilized reagent of Example 1 was used and as the apparatus for fibrinogen determination, the CG02N blood coagulation analyzer (A&T Corporation) was used to examine specificity and reproducibility of the fibrinogen concentration in plasma.

The reagent was mounted on the CG02N analyzer, and 25 μl of a plasma sample with known fibrinogen concentration was added thereto to determine the clotting time. 4 types of plasma samples were each subjected to the procedure 5 times. According to the results obtained in Example 1, the calibration curve of the lyophilized reagent indicates LN (fibrinogen concentration)=−0.7606×LN (clotting time)+7.01. Thus, the determined clotting time was converted to the fibrinogen concentration with the formula below.

Fibrinogen concentration in citrated plasma=e^{7.01}×(clotting time)^{-0.7606} [Formula 5]

Specificity was evaluated based on the recovery rate relative to the known fibrinogen concentration, and reproducibility was evaluated based on the CV value (coefficient of variation) obtained by 5 continuous measurements.

The results are shown in Table 2. As is apparent from Table 2, specificity and reproducibility were observed in the fibrinogen concentration.

TABLE 2

| | Fib concentration | | | |
|---|---|---|---|---|
| Number of assays | 31 mg/dl | 46 mg/dl | 107 mg/dl | 140 mg/dl |
| First | 34 | 48 | 106 | 158 |
| Second | 32 | 49 | 104 | 136 |
| Third | 33 | 51 | 106 | 140 |
| Fourth | 35 | 60 | 95 | 155 |
| Fifth | 32 | 50 | 99 | 133 |
| Average (mg/dl) | 33 | 52 | 102 | 144 |
| Specificity (%) | 107 | 112 | 95 | 103 |
| CV (%) | 4.4 | 9.0 | 4.7 | 8.0 |

### Example 3: Correlation Between Clauss's Method and the Method Using the Fibrinogen Measurement Dry Reagent According to the Present Disclosure

The correlation between the results of fibrinogen determination by Clauss's method and the results of fibrinogen determination using the fibrinogen measurement dry reagent according to the present disclosure was examined using 51 human plasma samples. Fibrinogen determination by Clauss's method was performed using the Data Fi fibrinogen reagent (Sysmex Corporation) and the KC4 Delta™ coagulation analyzer (Tcoag Ireland Ltd.) by the method described in the package insert attached to the Data Fi fibrinogen reagent.

Fibrinogen determination with the fibrinogen measurement dry reagent according to the present disclosure was performed with the lyophilized reagent of Example 1 as the fibrinogen measurement dry reagent and the CG02N blood coagulation analyzer (A&T Corporation) as the apparatus for determining fibrinogen.

The lyophilized reagent was mounted on the CG02N analyzer, 25 μl of the samples was added thereto, and the clotting time of each sample was obtained by the method described above. The obtained clotting time was converted to the fibrinogen concentration using Formula 5.

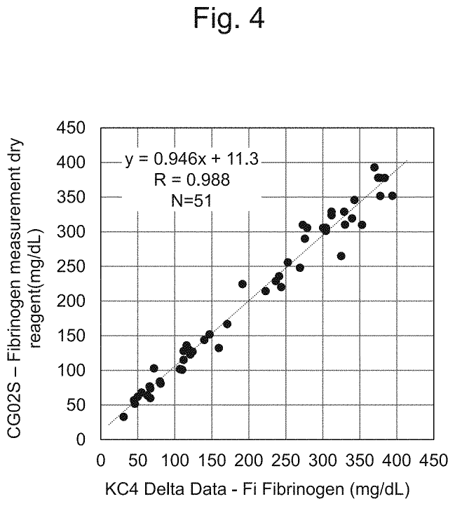

FIG. 4 shows the correlation between the quantitative value of fibrinogen determined by Clauss's method and the quantitative value of fibrinogen determined with the fibrinogen measurement dry reagent according to the present disclosure. As is apparent from FIG. 4, the quantitative value of fibrinogen determined with the fibrinogen measurement dry reagent according to the present disclosure is very consistent and highly correlated with the quantitative value of fibrinogen determined by the Clauss method.

### Example 4: Correlation Between Citrated Plasma Samples and Citrated Whole Blood Samples

51 citrated whole blood samples were subjected to fibrinogen determination with the fibrinogen measurement dry reagent according to the present disclosure, 51 citrated plasma samples obtained via centrifugation of the 51 citrated whole blood samples were subjected to fibrinogen determination with the fibrinogen measurement dry reagent according to the present disclosure, and the correlation between these results of fibrinogen determination was examined. The composition of the fibrinogen measurement dry reagent according to the present disclosure was as follows:

160 μg/mil polybrene

2.5 (wt/v) % glycine

10 mM CaCl₂.2H₂O

1.2 mg/ml bovine serum albumin

0.005 (wt/v) % sorbitan monolaurate

200 μg/ml GPRP-amide

40 mM HEPES-NaOH buffer (pH 7.35)

333 NIHU/ml bovine thrombin

The apparatus and the procedure employed herein were identical to those in Example 3. Since the calibration curve of the lyophilized reagent indicates: LN (fibrinogen concentration)=−0.7636×LN (clotting time)+7.22, the determined clotting time was converted to the fibrinogen concentration with the formula below.

Fibrinogen concentration in citrated plasma=e^{7.22}×(clotting time)^{-0.7636} [Formula 6]

When citrated whole blood was used as the measurement sample, the fibrinogen concentration in the sample was determined in the following manner. First, hematocrit values of the 51 citrated whole blood assay samples were determined using the blood cell counter MYTHIC22 (J) (A&T Corporation). Subsequently, the lyophilized reagent was mounted on the CG02N blood coagulation analyzer (A&T Corporation), the assay mode was changed to whole blood assay mode, 25 μl of the citrated whole blood was added thereto, and the clotting time of each sample was then determined.

The clotting time was converted to the fibrinogen concentration using Formula 6, and the fibrinogen concentration in the citrated whole blood sample was determined using Formula 4.

When citrated plasma was used as the measurement sample, the fibrinogen concentration of the sample was determined in the manner described below. First, 51 citrated whole blood assay samples were centrifuged at 4° C. and 3,000 rpm for 15 minutes, and 51 citrated plasma samples were obtained from the supernatant. Subsequently, the lyophilized reagent above was mounted on the CG02N analyzer, the assay mode was changed to plasma assay mode, 25 μl of the citrated plasma was added, and the clotting time of each sample was determined. The clotting time was converted to the fibrinogen concentration using Formula 6.

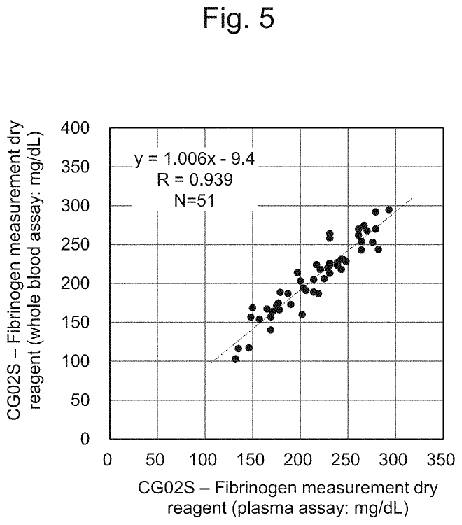

FIG. 5 shows the correlation between the quantitative value of fibrinogen in the citrated plasma measurement samples and the quantitative value of fibrinogen in the citrated whole blood measurement samples determined with the fibrinogen measurement dry reagent according to the present disclosure. As is apparent from FIG. 5, the quantitative value of fibrinogen in the citrated whole blood sample is very consistent and highly correlated with the quantitative value of fibrinogen in the citrated plasma samples determined with the fibrinogen measurement dry reagent according to the present disclosure.

### Example 5: Preparation of Reagents at Various Glycine Concentrations and Evaluation Thereof

The effects of the glycine content in the fibrinogen measurement dry reagent were examined in terms of the clotting time of the citrated plasma and the clotting times of the citrated whole blood and simultaneous reproducibility thereof. First, the reagent composition as used in Example 4 was used to prepare lyophilized reagents although the glycine concentration of the composition of the reagent was set as 0.5%, 1.0%, 1.5%, 2.0%, 2.5%, 3.0%, 3.5%, 4.0%, 4.5%, and 5.0% for each sample. Subsequently, citrated plasma with a fibrinogen concentration of 181 mg/dl was continuously measured using the lyophilized reagents on the CG02N analyzer, and the clotting time and the CV values obtained by the 5 continuous measurements were recorded.

TABLE 3

| | Present disclosure | | | | | | | | | |
|---|---|---|---|---|---|---|---|---|---|---|
| | Glycine concentration | | | | | | | | | |
| Plasma | 0.5% | 1.0% | 1.5% | 2.0% | 2.5% | 3.0% | 3.5% | 4.0% | 4.5% | 5.0% |
| 1 (s) | 13.9 | 13.4 | 11.1 | 10.7 | 9.4 | 7.5 | 6.2 | 6.5 | 6.0 | 4.4 |
| 2 (s) | 14.5 | 14.7 | 10.7 | 10.4 | 9.8 | 6.4 | 7.4 | 6.6 | 5.7 | 4.7 |
| 3 (s) | 13.9 | 15.3 | 10.8 | 10.9 | 8.6 | 8.4 | 6.2 | 6.8 | 4.9 | 4.5 |
| 4 (s) | 14.7 | 13.8 | 10.6 | 11.2 | 10.3 | 7.4 | 7.2 | 7.1 | 6.0 | 5.1 |
| 5 (s) | 12.6 | 15.1 | 11.2 | 9.7 | 9.2 | 7.2 | 8.0 | 6.7 | 5.5 | 5.1 |
| AVG (s) | 13.9 | 14.5 | 10.9 | 10.6 | 9.5 | 7.4 | 7.0 | 6.7 | 5.6 | 4.8 |
| SD | 0.8 | 0.8 | 0.3 | 0.6 | 0.6 | 0.7 | 0.8 | 0.2 | 0.5 | 0.3 |
| CV (%) | 5.9 | 5.7 | 2.4 | 5.4 | 6.8 | 9.7 | 11.2 | 3.4 | 8.1 | 6.9 |

(s): Clotting time; AVG: average; SD: standard deviation; CV: Coefficient of variation

As shown in Table 3, when the glycine concentration in the reagent is less than 1.5%, the clotting time is extremely prolonged because of the lack of reagent solubility. However, when the glycine concentration in the reagent is 1.5% or higher, solubility is enhanced, and a shortened clotting time is obtained. When the glycine concentration in the reagent is over 4.5%, the clotting time determined by the CG02N blood coagulation analyzer is lower than the lower detection limit, which is 5.0 seconds. This indicates that it is not possible to perform fibrinogen quantification of a sample with a fibrinogen concentration exceeding 181 mg/dl. In other words, in the case of a reagent with the glycine concentration exceeding 4.5%, it is not possible to determine whether or not the fibrinogen concentration in the sample has returned within the normal range (200 to 400 mg/dl) as a result of administration of a fibrinogen preparation. Therefore, in the case of plasma measurements, it is apparent that the glycine concentration in the reagent is preferably within the range of 1.5% to 4.0%.

Subsequently, citrated whole blood assay samples with a fibrinogen concentration of 181 mg/dl were continuously measured 5 times using the lyophilized reagents on the CG02N analyzer, and the clotting time and the CV values obtained by the 5 continuous measurements were recorded.

TABLE 4

| | Present disclosure | | | | | | | | | |
|---|---|---|---|---|---|---|---|---|---|---|
| | Glycine concentration | | | | | | | | | |
| Whole blood | 0.5% | 1.0% | 1.5% | 2.0% | 2.5% | 3.0% | 3.5% | 4.0% | 4.5% | 5.0% |
| 1 (s) | 46.9 | 52.1 | 30.6 | 25.3 | 20.7 | 17.3 | 15.4 | 12.2 | 10.2 | 5.6 |
| 2 (s) | 39.4 | 49.5 | 32.6 | 24.1 | 18.7 | 18.5 | 14.2 | 12.1 | 12.4 | 6.9 |
| 3 (s) | 39.3 | 58.9 | 27.7 | 27.7 | 22.4 | 16.2 | 15.2 | 11.9 | 10.3 | 7.3 |
| 4 (s) | 58.0 | 65.5 | 34.5 | 23.2 | 22.5 | 17.3 | 16.0 | 9.8 | 11.4 | 8.1 |
| 5 (s) | 47.2 | 55.1 | 37.8 | 26.4 | 22.1 | 14.9 | 15.0 | 9.0 | 11.1 | 9.0 |
| AVG (s) | 46.2 | 56.2 | 32.6 | 25.3 | 21.3 | 16.8 | 15.2 | 11.0 | 11.1 | 7.4 |
| SD | 7.7 | 6.3 | 3.8 | 1.8 | 1.6 | 1.4 | 0.7 | 1.5 | 0.9 | 1.3 |
| CV (%) | 16.6 | 11.1 | 11.7 | 7.1 | 7.6 | 8.1 | 4.3 | 13.6 | 8.1 | 17.3 |

(s): Clotting time; AVG: average; SD: standard deviation; CV: Coefficient of variation

As shown in Table 4, the clotting time is extremely prolonged because of a lack of reagent solubility when the glycine concentration in the reagent is less than 1.5%. On the other hand, when the glycine concentration in the reagent is 1.5% or higher, solubility is enhanced, and a shortened clotting time is obtained. In the case of whole blood measurements, accordingly, it is apparent that the glycine concentration in the reagent is preferably 1.5% or higher.

### Comparative Example 1: Comparison of Properties with Lyophilized Reagent of Conventional Composition

Properties of the fibrinogen measurement dry reagent according to the present disclosure were compared with those of a lyophilized reagent prepared with the reagent composition described in JP Patent No. 3469909.

A fibrinogen measurement dry reagent with the glycine concentration of 2.5% was prepared by the method described in Example 1. Also, a lyophilized reagent having the composition described below was prepared by the method described in Example 1. The reagent composition is reported in JP H05-219993 A (JP Patent No. 3469909).

### Reagent Composition of Comparative Example

15 μg/ml polybrene

10 mM CaCl₂.2H₂O

1.0 (wt/v) % bovine serum albumin

0.08 (wt/v) % Polyethylene glycol 6000

200 μg/ml polymerization inhibitor (GPRP-amide)

50 mM Tris-HCl buffer (pH8.0)

50 IU/ml bovine thrombin

110 mM NaCl

The citrated plasma samples and the citrated whole blood samples with the fibrinogen concentration of 162 mg/dl were continuously measured 5 times using the relevant reagents on the CG02N analyzer, and the clotting time and the CV values obtained by the 5 continuous measurements were recorded. Also, changes in the movement signal of the magnetic particles detected with the elapse of time in measurements were recorded.

TABLE 5

| | Plasma assay | | Whole blood assay | |
|---|---|---|---|---|
| | Dry | | Dry | |
| | reagent for | | reagent for | |
| | fibrinogen | Lyophilized | fibrinogen | Lyophilized |
| | determination | reagent of | determination | reagent of |
| Number | of the present | conventional | of the present | conventional |
| of assays | disclosure | composition | disclosure | composition |
| First (sec) | 10.5 | 31.6 | 25.6 | 54.1 |
| Second (sec) | 9.8 | 32.4 | 24.4 | 45.7 |
| Third (sec) | 11.1 | 35.4 | 24.0 | 62.9 |
| Fourth (sec) | 11.0 | 29.8 | 23.4 | 54.2 |
| Fifth (sec) | 11.1 | 32.6 | 24.8 | 77.4 |
| Average (sec) | 10.7 | 32.4 | 24.4 | 58.9 |
| Standard | 0.6 | 2.0 | 0.8 | 12.0 |
| deviation | | | | |
| CV (%) | 5.2 | 6.3 | 3.4 | 20.4 |

CV: Coefficient of variation

As shown in Table 5, it is clear that the clotting time obtained with the fibrinogen measurement dry reagent according to the present disclosure is shorter than that obtained with a lyophilized reagent of a conventional composition, and accordingly, the reproducibility of the clotting time is good.

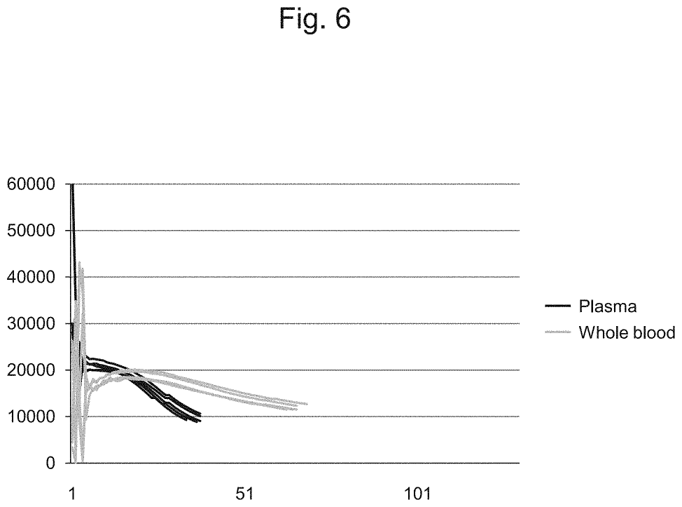

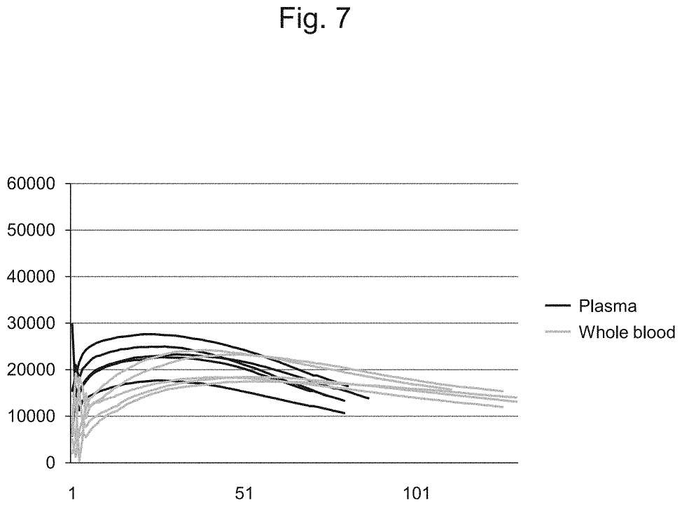

FIG. 6 and FIG. 7 show changes in the movement signal of the magnetic particles detected with the elapse of time in the measurements. FIG. 6 shows a chart demonstrating changes in the movement signal of the magnetic particles with the elapse of time when measured with the fibrinogen measurement dry reagent according to the present disclosure. FIG. 7 shows a chart demonstrating changes in the movement signal of the magnetic particles with the elapse of time when measured with the lyophilized reagent prepared with the reagent composition of the conventional technique. In the charts, the horizontal axis indicates the time elapsed after the sample is added, a numerical value “51” indicates 25.5 seconds, and a numerical value “101” indicates 50.5 seconds. The vertical axis indicates the amount of change in scattered light; i.e., the movement signal of the magnetic particles (unit: counts). Changes in the movement signal of the magnetic particles with the elapse of time were more constant among the 5 measurements conducted with the fibrinogen measurement dry reagent according to the present disclosure and it is clear that the movement signal of the magnetic particles are attenuated to a significant extent as the clotting reaction proceeds. In contrast, changes in the movement signal of the magnetic particles with the elapse of time varied significantly among the 5 measurements conducted with the lyophilized reagent of the conventional composition, and the attenuation of the movement signal of the magnetic particles as the clotting reaction proceeds is moderate. When such reagent is used, there is a risk of erroneous measurement.

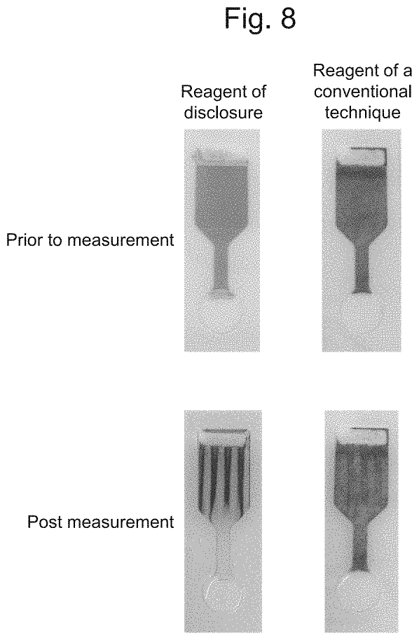

FIG. 8 shows photographs of the reagents before and after plasma measurements. In FIG. 8, the upper photographs show reagents before the measurements and the lower photographs show the reagents after the measurements. In the case of the lyophilized reagent of the conventional composition, reagent solubility is insufficient and, therefore, magnetic particles are aggregated locally after the measurements, and it is difficult to identity magnetic particle lines (beams) derived from the permanent magnetic field are difficult to identify. This means that the movement of magnetic particles does not necessarily correspond to the change in the viscosity in the reaction system caused as the coagulation reaction proceeds. In contrast, in the case of the reagent of the present disclosure (glycine concentration in the reagent: 2.5%), reagent solubility is improved, and the magnetic particle lines derived from the permanent magnetic field can clearly be distinguished. In the same manner, magnetic particle lines derived from the permanent magnetic field could also clearly be identified with the reagent of the present disclosure with the glycine concentration of 1.5%, 2.0%, 3.0%, 3.5%, or 4.0%. Concerning the reagents with the glycine concentration of 4.5% and 5.0%, local polymerization of magnetic particles was observed and the appearance of the particles was not always good after the measurements.

### Example 6: Measurement of Clotting Time by the Fibrinogen Determination Method According to the Present Disclosure

First, in accordance with Example 1, the fibrinogen measurement dry reagent was prepared in the manner described below.

A 40 mM HEPES buffer (pH 7.35) supplemented with 10 mM CaCl₂.2H₂O, 2.0 (wt/v) % glycine, 160 μg/ml polybrene, 1.2 mg/ml bovine serum albumin, 0.005 (wt/v) % sorbitan monolaurate, and 200 μg/ml GPRP-amide was added to a lyophilized bovine thrombin product (Oriental Yeast Co., Ltd.) and dissolved to obtain a reagent solution having 333 NIHU/ml of thrombin activity. To 35 ml of the reagent solution, 0.47 g of triiron tetraoxide (product name: AAT-03; average particle diameter: 0.35 μm: Toda Kogyo Corp.) was added and suspended to obtain a final solution. The final solution (25 μl) was dispensed onto the reaction slide shown in FIG. 1. The reaction slide was stored and frozen in a freezer maintained at −40° C. for one whole day and night. Subsequently, the frozen reaction slide was lyophilized. Lyophilization was performed under the conditions in which the temperature was linearly raised from −30° C. to −20° C. over a period of 24 hours in vacuum, the temperature was linearly raised from −20° C. to 30° C. over a period of 20 hours, the temperature was maintained at 30° C. for 3 hours, and then dry air was applied to release the vacuum. The lyophilized reagent was immediately sealed with an aluminum film in a dehumidified environment.

Whole blood assay samples were measured using the fibrinogen measurement dry reagent described above by the fibrinogen determination method according to the present disclosure. According to this method, monitoring of the movement signal of the magnetic particles was initiated immediately after the samples were added and monitoring was performed at an interval of 0.5 seconds. That is, the period for monitoring the movement signal of the magnetic particles is 0.5 seconds. The ratio of the movement signals of the magnetic particles was then continuously computed at an interval of 1 second. In other words, the time interval used to compute the ratio of the movement signals of the magnetic particles is 1 second. That is, the ratio of the movement signals of the magnetic particles is computed as follows: (the movement signal of the magnetic particles detected at the monitoring time of 1.0 second)/(the movement signal of the magnetic particles detected at the monitoring time of 0 seconds), (the movement signal of the magnetic particles detected at the monitoring time of 1.5 seconds)/(the movement signal of the magnetic particles detected at the monitoring time of 0.5 seconds), (the movement signal of the magnetic particles detected at the monitoring time of 2.0 seconds)/(the movement signal of the magnetic particles detected at the monitoring time of 1.0 second) . . . . The interval during which the ratio was maintained within a range of 1.0±0.1 for 1.5 seconds was the period of 5.0 to 6.5 seconds in terms of the monitoring time. The first point thereof was at the monitoring time of 5.0 seconds, and this point was thus designated as the starting point (the starting point of the coagulation reaction: measurement time 0 (sec)). The peak value of the movement signal of the magnetic particles at or after the starting point was 2726c detected at the monitoring time of 7.0 seconds. The movement signal of the magnetic particles that was lower by 30% from the peak value of the movement signal of the magnetic particles at or after the starting point was computed to be 1908c. That is, the end point was the point at which the movement signal of the magnetic particles was 1908c, and the clotting time was computed to be 20.1 seconds. The results are shown in Table 6.

TABLE 6

| Time point | | Ratio of movement | | |
|---|---|---|---|---|
| for | | signal of magnetic | | |
| monitoring | | particle at the time | | |
| movement | Movement | interval of 1 sec for | | |
| signal of | signal of | calculating the rate | | |
| the magnetic | magnetic | of movement signals | Start/ | Measurement |
| particles | particles | of the magnetic | peak/ | time |
| (sec) | (C) | particles (−) | end | (sec) |
| 0 | 1430 | — | | |
| 0.5 | 86 | — | | |
| 1.0 | 359 | 0.25 | | |
| 1.5 | 114 | 1.33 | | |
| 2.0 | 3722 | 10.37 | | |
| 2.5 | 4235 | 37.15 | | |
| 3.0 | 1841 | 0.49 | | |
| 3.5 | 3534 | 0.83 | | |
| 4.0 | 2890 | 1.57 | | |
| 4.5 | 2389 | 0.68 | | |
| 5.0 | 2673 | 0.92 | Start | 0 |
| 5.5 | 2581 | 1.08 | | 0.5 |
| 6.0 | 2682 | 1.00 | | 1.0 |
| 6.5 | 2651 | 1.03 | | 1.5 |
| 7.0 | 2726 | 1.02 | Peak | 2.0 |
| 7.5 | 2678 | 1.01 | | 2.5 |
| 3.0 | 2721 | 1.00 | | 3.0 |
| 8.5 | 2673 | 1.00 | | 3.5 |
| 9.0 | 2708 | 1.00 | | 4.0 |
| 9.5 | 2665 | 1.00 | | 4.5 |
| 10.0 | 2677 | 0.99 | | 5.5 |
| 10.5 | 2635 | 0.99 | | 6.0 |
| 11.0 | 2635 | 0.98 | | 6.5 |
| . | . | . | | . |
| . | . | . | | . |
| . | . | . | | . |
| 22.0 | 2031 | 0.98 | | 17.5 |
| 22.5 | 2007 | 0.98 | | 18.0 |
| 23.0 | 1980 | 0.97 | | 18.5 |
| 23.5 | 1957 | 0.98 | | 19.0 |
| 24.0 | 1940 | 0.98 | | 19.5 |
| 24.5 | 1913 | 0.98 | | 20.0 |
| — | 1908 | — | end | 20.1 |
| 25.0 | 1889 | 0.97 | | 20.5 |
| 25.5 | 1870 | 0.98 | | 21.0 |

Peak movement signal of magnetic particles after the starting point: 2726
Movement signal of magnetic particles attenuated by 30% from the peak movement signal of magnetic particles after the starting point: 1908
Clotting time = 20.1 sec

### Example 7: Comparison of the Conventional Fibrinogen Determination Method (the Determination Method According to JP Patent No. 2980468) with the Fibrinogen Determination Method According to the Present Disclosure (the Present Disclosure) when Undiluted Whole Blood Samples are Measured Using the Fibrinogen Measurement Dry Reagent

The fibrinogen measurement dry reagent was prepared in the manner described above.

First, the calibration curve according to the conventional determination method (the determination method according to JP Patent No. 2980468) was set up. The calibration curve was set up in the manner described below. Human plasma containing 304 mg/dl of fibrinogen and fibrinogen-deficient plasma (Clinisys Associate) were used to prepare 7 serial dilution samples of human plasma from 37 to 304 mg/dl. Subsequently, the fibrinogen measurement dry reagent was mounted on the CG02N blood coagulation analyzer (A&T Corporation), 25 μl each of the serial dilution samples were added thereto, and the clotting time of each sample was obtained. Finally, the data were plotted by setting the Y axis to LN (fibrinogen concentration) and the X axis to LN (clotting time), and determining the regression formula to compute the calibration curve according to the conventional determination method.

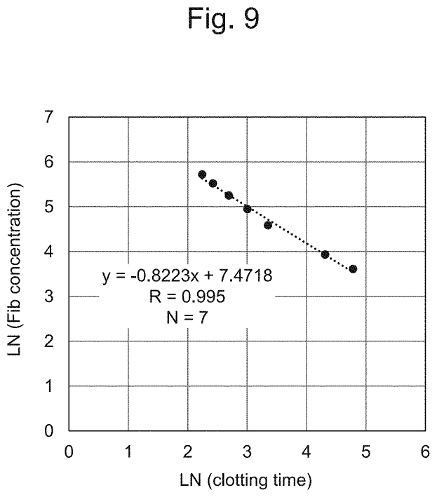

As a result, the calibration curve according to the conventional determination method was found to be as follows (FIG. 9):

LN(fibrinogen concentration)=−0.8223×LN(clotting time)+7.4718 [Formula 7]

Based on the calibration curve formula above, the fibrinogen concentration conversion formula shown below was employed.

Fibrinogen concentration in a sample=e^{7.4718}×(clotting time)^{-0.8223} [Formula 8]

Subsequently, the calibration curve according to the determination method of the present disclosure was set up. The calibration curve was set up in the manner described below. Human plasma containing 304 mg/dl of fibrinogen and fibrinogen-deficient plasma (Clinisys Associate) were used to prepare 7 serial dilution samples of human plasma from 37 to 304 mg/dl. Subsequently, the fibrinogen measurement dry reagent was mounted on the CG02N blood coagulation analyzer (A&T Corporation), the software of the present disclosure was integrated therein, 25 μl each of the serial dilution samples was added thereto, and the clotting time of each sample was determined. Finally, the data were plotted by setting the Y axis to LN (fibrinogen concentration) and the X axis to LN (clotting time), and by determining the regression formula, the calibration curve according to the determination method of the present disclosure was computed.

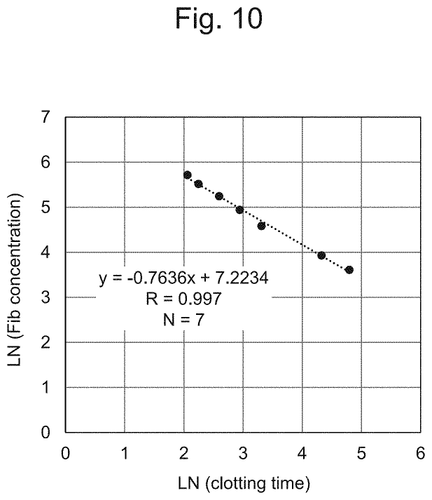

As a result, the calibration curve according to the determination method of the present disclosure was found to be as follows (FIG. 10):

LN(fibrinogen concentration)=−0.7636×LN(clotting time)+7.2234 [Formula 9]

Based on the calibration curve formula above, the fibrinogen concentration conversion formula shown below was employed.

Fibrinogen concentration in sample=e^{7.2234}×(clotting time)^{-0.7636} [Formula 10]

Blood samples were obtained from one healthy subject using 7 blood collection tubes with sodium citrate (2 ml) to obtain 14 ml of citrated whole blood samples. The 7 blood collection tubes were subjected to centrifugation at 4° C. and 3,000 rpm for 15 minutes. Among the seven centrifuged blood collection tubes, three tubes were set aside, and the supernatants (plasma samples) from the 4 blood collection tubes were aliquoted in amounts of 1 ml each and dispensed into PP containers to obtain 4 ml of citrated plasma sample A. Citrated plasma sample A (2.80 ml) was added to 1 out of the 3 remaining collection tubes which were set aside, and the collection tube was hermetically sealed, followed by mixing by inversion to obtain citrated whole blood sample B. Separately, citrated plasma sample A (0.40 ml) was added to (another) 1 out of the remaining 3 collection tubes set aside, and the collection tube was hermetically sealed, followed by mixing by inversion to obtain citrated whole blood sample C. Further, 0.56 ml of the supernatant (plasma sample) was removed from 1 out of the remaining 3 collection tubes set aside, and the collection tube was hermetically sealed, followed by mixing by inversion to obtain citrated whole blood sample D.

The hematocrit values of citrated whole blood sample B, citrated whole blood sample C, and citrated whole blood sample D were determined using the blood cell counter MYTHIC22 (J) (distributed by A&T Corporation). As a result, the hematocrit value of citrated whole blood sample B was 15%, that of citrated whole blood sample C was 30%, and that of citrated whole blood sample D was 50%.

Fibrinogen concentrations in citrated plasma sample A, citrated whole blood sample B, citrated whole blood sample C, and citrated whole blood sample D were examined according to the conventional determination method (JP Patent No. 2980468).

The fibrinogen measurement dry reagent was mounted on the CG02N analyzer, the assay mode was changed to plasma assay mode, 25 μl of citrated plasma sample A was added, and the clotting time was obtained. This procedure was carried out 5 times. The obtained clotting time was applied to the conversion formula mentioned above (i.e., fibrinogen concentration in sample=e^{7.4718}×(clotting time)^{-0.8223}) to determine the fibrinogen concentration in citrated plasma sample A according to the conventional determination method.

The fibrinogen measurement dry reagent was mounted on the CG02N analyzer, the assay mode was changed to whole blood assay mode, 25 μl of citrated whole blood sample B was added thereto, and the clotting time was obtained. This procedure was carried out 5 times. The obtained clotting time was applied to the same conversion formula and converted into the fibrinogen concentration. In addition, the fibrinogen concentration in citrated whole blood sample B according to the conventional determination method was determined with the formula shown below.

Fibrinogen concentration in citrated whole blood sample B according to the conventional determination method=converted fibrinogen concentration×(100/(100−15)) [Formula 11]

The fibrinogen measurement dry reagent was mounted on the CG02N analyzer, the assay mode was changed to whole blood assay mode, 25 μl of citrated whole blood sample C was added thereto, and the clotting time was obtained. This procedure was carried out 5 times. The obtained clotting time was applied to the same conversion formula and converted into the fibrinogen concentration. In addition, the fibrinogen concentration in citrated whole blood sample C according to the conventional determination method was determined with the formula shown below.

Fibrinogen concentration in citrated whole blood sample C according to the conventional determination method=converted fibrinogen concentration×(100/(100−30)) [Formula 12]

The fibrinogen measurement dry reagent was mounted on the CG02N analyzer, the assay mode was changed to whole blood assay mode, 25 μl of citrated whole blood sample D was added thereto, and the clotting time was obtained. This procedure was carried out 5 times. The obtained clotting time was applied to the same conversion formula and converted into the fibrinogen concentration. In addition, the fibrinogen concentration in citrated whole blood sample D according to the conventional determination method was determined with the formula shown below.

Fibrinogen concentration in citrated whole blood sample D according to the conventional determination method=converted fibrinogen concentration×(100/(100−50)) [Formula 13]

Subsequently, fibrinogen concentrations in citrated plasma sample A, citrated whole blood sample B, citrated whole blood sample C, and citrated whole blood sample D were examined according to the determination method of the present disclosure.

The fibrinogen measurement dry reagent was mounted on the CG02N analyzer, the software that implements the fibrinogen determination method according to the present disclosure was integrated therein, the assay mode was changed to plasma assay mode, 25 μl of citrated plasma sample A was added, and the clotting time was obtained. This procedure was carried out 5 times. The obtained clotting time was applied to the conversion formula mentioned above (i.e., fibrinogen concentration in sample=e^{-7.2234}×(clotting time)^{-0.7636}) to determine the fibrinogen concentration in citrated plasma sample A according to the determination method of the present disclosure.

The fibrinogen measurement dry reagent was mounted on the CG02N analyzer, the software that implements the fibrinogen determination method according to the present disclosure was integrated therein, the assay mode was changed to whole blood assay mode, 25 μl of citrated whole blood sample B was added, and the clotting time was obtained. This procedure was carried out 5 times. The obtained clotting time was applied to the same conversion formula and converted into the fibrinogen concentration. In addition, the fibrinogen concentration in citrated whole blood sample B according to the determination method of the present disclosure was determined with the formula shown below.

Fibrinogen concentration in citrated whole blood sample B according to the determination method of the present disclosure=converted fibrinogen concentration×(100/(100−15)) [Formula 14]

The fibrinogen measurement dry reagent was mounted on the CG02N analyzer, the software that implements the fibrinogen determination method according to the present disclosure was integrated therein, the assay mode was changed to whole blood assay mode, 25 μl of citrated whole blood sample C was added, and the clotting time was obtained. This procedure was carried out 5 times. The obtained clotting time was applied to the same conversion formula and converted into the fibrinogen concentration. In addition, the fibrinogen concentration in citrated whole blood sample C according to the determination method of the present disclosure was determined with the formula shown below.

Fibrinogen concentration in citrated whole blood sample C according to the determination method of the present disclosure=converted fibrinogen concentration×(100/(100−30)) [Formula 15]

The fibrinogen measurement dry reagent was mounted on the CG02N analyzer, the software that implements the fibrinogen determination method according to the present disclosure was integrated therein, the assay mode was changed to whole blood assay mode, 25 μl of citrated whole blood sample D was added, and the clotting time was obtained. This procedure was carried out 5 times. The obtained clotting time was applied to the same conversion formula and converted into the fibrinogen concentration. In addition, the fibrinogen concentration in citrated whole blood sample D according to the determination method of the present disclosure was determined with the formula shown below.

Fibrinogen concentration in citrated whole blood sample D according to the determination method of the present disclosure=converted fibrinogen concentration×(100/(100−50)) [Formula 16]

In addition, citrated plasma sample A was determined by the Clauss method. Fibrinogen determination by Clauss's method was performed using the Data Fi fibrinogen reagent (Sysmex Corporation) and the KC4 Delta™ coagulation analyzer (Tcoag Ireland Ltd.) by the method described in the package insert attached to the Data Fi fibrinogen reagent. Fibrinogen determination was performed 5 times, and the average value thereof (i.e., 224 mg/dl) was determined to be the fibrinogen concentration in citrated plasma sample A by the Clauss method. The results are shown below.

TABLE 7

| | Fib concentration | | | |
|---|---|---|---|---|
| | Citrated | Citrated whole | Citrated whole | Citrated whole |
| Number | plasma | blood B | blood C | blood D |
| of assays | A | Ht value = 15% | Ht value = 30% | Ht value = 50% |
| First | 231 | 229 | 229 | 259 |
| Second | 219 | 222 | 239 | 263 |
| Third | 219 | 235 | 241 | 233 |
| Fourth | 223 | 232 | 216 | 278 |
| Fifth | 231 | 219 | 209 | 257 |
| Average | 225 | 228 | 227 | 258 |
| (mg/dl) | | | | |
| Specificity | 100.4 | 101.8 | 101.3 | 115.2 |
| (%) | | | | |
| CV (%) | 2.7 | 2.9 | 6.2 | 6.2 |

TABLE 8

| | Fib concentration | | | |
|---|---|---|---|---|
| | Citrated | Citrated whole | Citrated whole | Citrated whole |
| Number | plasma | blood B | blood C | blood D |
| of assays | A | Ht value = 15% | Ht value = 30% | Ht value = 50% |
| First | 235 | 229 | 227 | 220 |
| Second | 223 | 214 | 238 | 234 |
| Third | 212 | 234 | 248 | 216 |
| Fourth | 224 | 232 | 215 | 229 |
| Fifth | 228 | 222 | 208 | 206 |
| Average | 224 | 226 | 227 | 221 |
| (mg/dl) | | | | |
| Specificity | 100.0 | 100.9 | 101.3 | 98.7 |
| (%) | | | | |
| CV (%) | 3.6 | 3.6 | 7.2 | 5.0 |

Table 7 shows the results of measurements according to the conventional determination method and Table 8 shows the results of measurements according to the determination method of the present disclosure. Specificity was evaluated based on the recovery rate relative to the fibrinogen concentration (224 mg/dl) of citrated plasma sample A determined by the Clauss method. The sample exhibiting a higher hematocrit value has higher viscosity. According to Table 7, the whole blood sample D with high viscosity shows higher values than the plasma sample A in all measurements. That is, from the results shown in Tables 7 and 8 it is clear that fibrinogen concentration of a whole blood sample with high hematocrit value cannot be quantified accurately according to the conventional determination method, whereas, according to the determination method of the present disclosure, fibrinogen concentration of a whole blood sample with high hematocrit value can be determined accurately.

### Example 8: Correlation Between the Quantitative Value of Fibrinogen Determined by Clauss's Method and the Quantitative Value of Fibrinogen Determined by the Determination Method of the Present Disclosure

The correlation between the results of fibrinogen determination by Clauss's method and the results of fibrinogen determination by the determination method of the present disclosure was examined using 104 citrated plasma samples. Fibrinogen determination by the determination method of the present disclosure was performed in the manner described below.

The fibrinogen measurement dry reagent was mounted on the CG02N analyzer, the software that implements the fibrinogen determination method according to the present disclosure was integrated therein, the assay mode was changed to plasma assay mode, 25 μl of the citrated plasma sample was added, and the clotting time was obtained. The obtained clotting time was applied to the conversion formula mentioned above (i.e., fibrinogen concentration in a sample=e^{7.2234}×(clotting time)^{-0.7636}) to convert the same into fibrinogen concentration and the converted fibrinogen concentration was designated as the fibrinogen concentration according to the determination method of the present disclosure.

Fibrinogen determination according to Clauss's method was performed using the Hemos IL Fib CXL reagent (LSI Medience Corporation) and the clinical laboratory system STACIA (LSI Medience Corporation). Determination was performed by the method described in the package insert attached to Hemos IL Fib CXL.

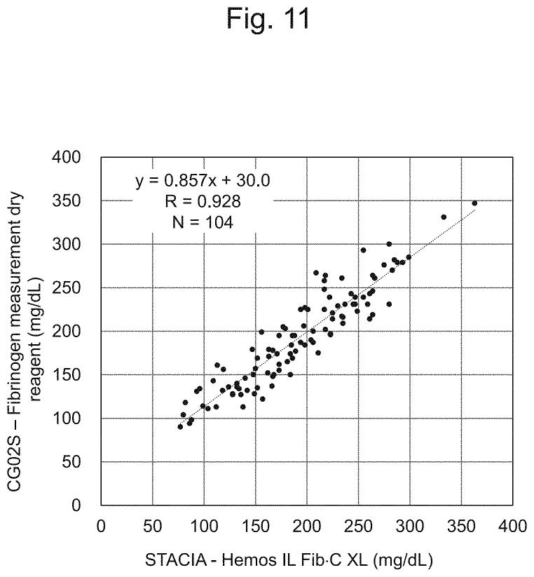

FIG. 11 shows the correlation between the quantitative value of fibrinogen determined by Clauss's method and the quantitative value of fibrinogen determined by the determination method of the present disclosure. As is apparent from FIG. 11, the quantitative value of fibrinogen determined by the determination method of the present disclosure is very consistent and highly correlated with the quantitative value of fibrinogen determined by the Clauss method.

### Example 9: Correlation Between the Quantitative Value of Fibrinogen Determined Using the Citrated Plasma Sample and the Quantitative Value of Fibrinogen Determined Using the Citrated Whole Blood Sample by the Method of the Present Disclosure

80 citrated whole blood assay samples were subjected to fibrinogen quantification by the determination method of the present disclosure, 80 citrated plasma samples obtained via centrifugation of the same 80 citrated whole blood assay samples were subjected to fibrinogen determination by the determination method of the present disclosure, and the correlation between these results of fibrinogen determination was examined.

First, hematocrit values of the 80 citrated whole blood assay samples were determined using the blood cell counter MYTHIC22 (J) (A&T Corporation). Subsequently, the fibrinogen measurement dry reagent was mounted on the CG02N analyzer. However, the software that implements the fibrinogen determination method according to the present disclosure was integrated therein, the assay mode was changed to whole blood assay mode, 25 μl of the citrated whole blood sample was added, and the clotting time of each sample was obtained. The obtained clotting time was applied to the conversion formula mentioned above, i.e.,

Fibrinogen concentration in sample=e^{7.2234}×(clotting time)^{-0.7636} [Formula 17]

to determine the fibrinogen concentration.

Finally, the fibrinogen concentration in the citrated whole blood assay sample was determined with the formula below.

Fibrinogen concentration in sample=converted fibrinogen concentration−(100/(100−hematocrit value)) [Formula 18]

The 80 citrated whole blood assay samples for which the above measurement was completed were subjected to centrifugation at 4° C. and 3,000 rpm for 15 minutes, and the supernatant was collected to obtain 80 citrated plasma samples. Subsequently, the fibrinogen measurement dry reagent was mounted on the CG02N analyzer. However, the software that implements the fibrinogen determination method according to the present disclosure was integrated therein, the assay mode was changed to plasma assay mode, 25 μl of the citrated plasma sample was added, and the clotting time of each sample was obtained. The obtained clotting time was applied to the conversion formula mentioned above, i.e.,

Fibrinogen concentration in sample=e^{-7.2234}×(clotting time)^{-0.7636} [Formula 19]

to convert the same into the fibrinogen concentration. The converted fibrinogen concentration was designated as the fibrinogen concentration in the citrated plasma assay sample.

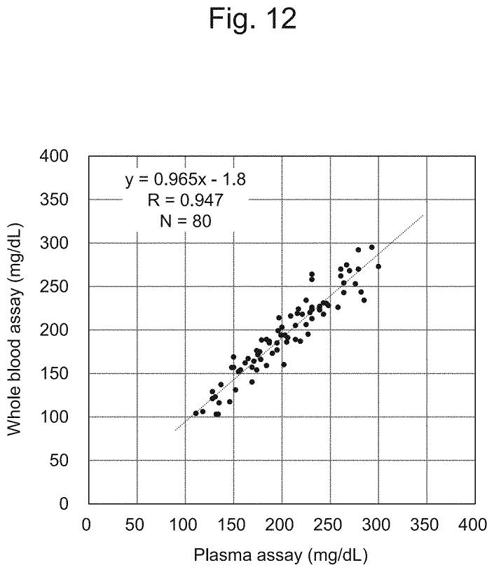

FIG. 12 shows the correlation between the quantitative value of fibrinogen determined using a citrated plasma assay sample and the quantitative value of fibrinogen determined using a citrated whole blood assay sample examined by the determination method according to the present disclosure. As is apparent from FIG. 12, the quantitative value of fibrinogen determined using a citrated whole blood assay sample is very consistent and highly correlated with the quantitative value of fibrinogen determined using a citrated plasma assay sample when the method of the present disclosure is employed.

### INDUSTRIAL APPLICABILITY

According to the present disclosure, fibrinogen determination can be performed without any dilution procedure.

All publications, patents, and patent applications cited herein are incorporated herein by reference in their entirety.

### DESCRIPTION OF REFERENCES

A: Transparent resin plate
B: Transparent resin plate
C: White resin plate
D: Reagent filling unit

1. A fibrinogen measurement dry reagent for use in measuring an undiluted whole blood or plasma sample comprising:
(i) thrombin or a protein having thrombin activity;
(ii) magnetic particles;
(iii) a fibrin monomer polymerization inhibitor;
(iv) a calcium salt;
(v) a dry reagent layer solubility improving agent;
(vi) a dry reagent layer reinforcing material; and
(vii) a buffer.

2. The fibrinogen measurement dry reagent of claim 1, wherein;
the thrombin or the protein having thrombin activity is bovine thrombin,
the magnetic particles are triiron tetraoxide particles,
the inhibitor of fibrin monomer polymerization is GPRP-amide or GHRP-amide, or
the calcium salt is calcium chloride dihydrate.

3.-5. (canceled)

6. The fibrinogen measurement dry reagent of claim 1, wherein the agent for improving solubility of the dry reagent layer is glycine.

7. The fibrinogen measurement dry reagent of claim 6, which comprises 1.5% to 4.0% by weight of glycine in the final solution.

8. The fibrinogen measurement dry reagent of claim 1, wherein the material for reinforcing the dry reagent layer is bovine serum albumin.

9. The fibrinogen measurement dry reagent of claim 1, wherein the pH buffer is HEPES-NaOH buffer.

10. The fibrinogen measurement dry reagent of claim 1, further comprising a heparin neutralizer and/or a defoaming agent,
wherein the heparin neutralizer is polybrene, and
wherein the defoaming agent is sorbitan monolaurate.

11. (canceled)

12. A fibrinogen determination method comprising:
(i) a step of adding a sample to a fibrinogen measurement dry reagent containing magnetic particles;
(ii) a step of allowing the magnetic particles in the reagent to move after the addition of the sample and monitoring the movement signal of the magnetic particles; and
(iii) a step of computing a plurality of ratios of the movement signals of the magnetic particles monitored in step (ii) at a given time interval,
wherein a point within an interval during which the ratio of the movement signals of the magnetic particles monitored at the given time interval is maintained within a given range for a given period of time is designated as the starting point, a point at or after the starting point at which the movement signal of the magnetic particles is attenuated by 5% to 50% from the peak value of the movement signal of the magnetic particles is designated as the end point, and the time from the starting point to the end point is designated as the clotting time.

13. The fibrinogen determination method of claim 12, wherein the time interval used to compute the ratio of the movement signals of the magnetic particles is a given time interval selected from between 0.1 seconds and 2 seconds.

14. The fibrinogen determination method of claim 12, wherein the time interval used to compute the ratio of the movement signals of the magnetic particles is a time interval of 0.5 seconds, 1 second, 1.5 seconds, or 2 seconds.

15. The fibrinogen determination method of claim 12, wherein the time interval used to compute the ratio of the movement signals of the magnetic particles is a time interval of 1 second.

16. The fibrinogen determination method of claim 12, wherein the given range of the ratio of the movement signals of the magnetic particles is 1.0±0.2.

17. The fibrinogen determination method of claim 13, wherein the given range of the ratio of the movement signals of the magnetic particles is 1.0±0.1.

18. The fibrinogen determination method of claim 12, wherein the time period during which the ratio of the movement signals of the magnetic particles is maintained within a given range is 1.5 seconds.

19. The fibrinogen determination method of claim 12, wherein the first point of the time period during which the ratio of the movement signals of the magnetic particles is maintained within a given range is designated as the starting point.

20. The fibrinogen determination method of claim 12, wherein a point at or after the starting point at which the movement signal of the magnetic particles is attenuated by 20% to 30% from the peak value of the movement signal of the magnetic particles is designated as the end point.

21. The fibrinogen determination method of claim 20, wherein a point at or after the starting point at which the movement signal of the magnetic particles is attenuated by 30% from the peak value of the movement signal of the magnetic particles is designated as the end point.

22. The fibrinogen determination method of claim 20, wherein a point at or after the starting point at which the movement signal of the magnetic particles is attenuated by 20% from the peak value of the movement signal of the magnetic particles is designated as the end point.

23. A program for executing the fibrinogen determination method of claim 12.

24. An information recording medium comprising the program of claim 23 recorded thereon.

25. An apparatus for fibrinogen determination comprising the program of claim 23 integrated therein.

26. An apparatus for fibrinogen determination comprising the information recording medium of claim 24 stored therein.
